SEQUENCE LISTING (85 sequences)

SEQ ID NO: 1 - protein, 451 aa, Porphyromonas gingivalis
MEIKEMVSLARKAQKEYQATHNQEAVDNICRAAAKVIYENAAILAREAVDETGMGVYEHKVAKNQGKSKGVWYNLHNKKSIGILNIDERTGMIEIAKPIGVVGAVTPTTNPIVTPMSNIIFALKTCNAIIIAPHPRSKKCSAHAVRLIKEAIAPFNVPEGMVQIIEEPSIEKTQELMGAVDVVVATGGMGMVKSAYSSGKPSFGVGAGNVQVIVDSNIDFEAAAEKIITGRAFDNGIICSGEQSIIYNEADKEAVFTAFRNHGAYFCDEAEGDRARAAIFENGAIAKDVVGQSVAFIAKKANINIPEGTRILVVEARGVGAEDVICKEKMCPVMCALSYKHFEEGVEIARTNLANEGNGHTCAIHSNNQAHIILAGSELTVSRIVVNAPSATTAGGHIQNGLAVTNTLGCGSWGNNSISENFTYKHLLNISRIAPLNSSIHIPDDKEIWEL

SEQ ID NO: 2 - DNA, 1356 bp, Porphyromonas gingivalis
atggagattaaagagatggtcagtcttgcgcgcaaagctcagaaggagtatcaggccacccataaccaagaagctgtggacaacatctgccgagcagcagcgaaggttatttacgaaaatgcagcaattctggcacgcgaggcagtggacgaaaccggcatgggtgtttacgagcacaaggtggccaagaatcaaggcaagtccaaaggtgtttggtacaacctgcataacaagaagtcgattggcatcctcaatatcgacgagcgtaccggcatgatcgagatcgcaaaacctatcggggttgtaggcgccgttacgccaaccaccaaccctatcgttactccgatgagcaacatcatctttgctcttaagacctgcaacgccatcattatcgccccacacccgcgctccaaaaagtgctctgcccacgcagttcggctgatcaaagaggctatcgctccgttcaacgtgcccgaaggtatggttcagatcatcgaggagcctagcatcgagaagacgcaggaattgatgggcgccgtagacgtggtcgttgctaccgggggcatgggcatggtcaagtctgcctactcctcagggaagccttctttcggtgtcggagccggcaatgttcaggtgatagtggacagcaacatcgacttcgaagcggcagcagaaaagatcatcaccggacgtgccttcgacaacggtatcatctgctcaggcgaacagtccatcatctacaacgaggctgacaaggaagcagttttcacagcattccgcaaccacggtgcgtacttttgcgacgaggccgagggagatcgggctcgtgcagcgatcttcgaaaatggagccatcgcgaaagatgttgtgggccagtccgttgcctttattgcaaagaaggcgaacattaatatccccgagggtactcgtattctcgtggtcgaagctcgcggagtaggcgccgaagatgtcatctgtaaagaaaagatgtgtccagtcatgtgcgccctctcctacaagcacttcgaagagggggtagagatcgcaaggacgaacctcgcaaacgaaggcaatggccatacctgtgctatccactccaacaaccaagcacacatcatcttggcaggctcggagctgaccgtgtctcgcatcgtggtcaacgcgccaagtgctaccacagcaggcggtcacatccagaacggtcttgccgtcaccaatactctaggctgcggctcttggggtaacaactcgatctccgaaaacttcacttataaacacctgctcaacatttcacgcatcgccccgttgaactccagcattcatatcccagatgataaggaaatctgggaactctaa

SEQ ID NO: 3 - protein, 314 aa, Corynebacterium glutamicum
MKETVGNKIVLIGAGDVGVAYAYALINQGMADHLAIIDIDEKKLEGNVMDLNHGVVWADSRTRVTKGTYADCEDAAMVVICAGAAQKPGETRLQLVDKNVKIMKSIVGDVMDSGFDGIFLVASNPVDILTYAVWKFSGLEWNRVIGSGTVLDSARFRYMLGELYEVAPSSVHAYIIGEHGDTELPVLSSATIAGVSLSRMLDKDPELEGRLEKIFEDTRDAAYHIIDAKGSTSYGIGMGLARITRAILQNQDVAVPVSALLHGEYGEEDIYIGTPAVVNRRGIRRVVELEITDHEMERFKHSANTLREIQKQFF

SEQ ID NO: 4 - protein, 490 aa, Corynebacterium glutamicum
MTINVSELLAKVPTGLLIGDSWVEASDGGTFDVENPATGETIATLASATSEDALAALDAACAVQAEWARMPARERSNILRRGFELVAERAEEFATLMTLEMGKPLAEARGEVTYGNEFLRWFSEEAVRLYGRYGTTPEGNLRMLTALKPVGPCLLITPWNFPLAMATRKVAPAIAAGCVMVLKPARLTPLTSQYFAQTMLDAGLPAGVLNVVSGASASAISNPIMEDDRLRKVSFTGSTPVGQQLLKKAADKVLRTSMELGGNAPFIVFEDADLDLAIEGAMGAKMRNIGEACTAANRFLVHESVADEFGRRFAARLEEQVLGNGLDEGVTVGPLVEEKARDSVASLVDAAVAEGATVLTGGKAGTGAGYFYEPTVLTGVSTDAAILNEEIFGPVAPIVTFQTEEEALRLANSTEYGLASYVFTQDTSRIFRVSDGLEFGLVGVNSGVISNAAAPFGGVKQSGMGREGGLEGIEEYTSVQYIGIRDPYAG

SEQ ID NO: 5 - protein, 453 aa, Corynebacterium glutamicum
MSLTFPVINPSDGSTITELENHDSTQWMSALSDAVAAGPSWAAKTPRERSVVLTAIFEALTERAQELAEIIHLEAGKSVAEALGEVAYGAEYFRWFAEEAVRLPGRYGQSPSGIGHIAVTRAPVGPVLAITPWNFPIAMATRKIAPALAAGCPVLVKPASETPLTMVKVGEIIASVFDTFNIPQGLVSIITTTRDAELSAELMADPRLAKVTFTGSTNVGRILVRQSADRLLRTSMELGGNAAFVIDEAADLDEAVSGAIAAKLRNAGQVCIAANRFLVHESRAAEFTSKLATAMQNTPIGPVISARQRDRIAALVDEAITDGARLIIGGEVPDGSGFFYPATILADVPAQSRIVHEEIFGPVATIATFTDLAEGVAQANSTEFGLAAYGFSNNVKATQYMAEHLEAGMVGINRGAISDPAAPFGGIGQSGFGREGGTEGIEEYLSVRYLALP

SEQ ID NO: 6 - protein, 521 aa, Corynebacterium glutamicum
MIKRLPLGPLPKELHQTLLDLTANAQDAAKVEVIAPFTGETLGFVFDGDEQDVEHAFALSRAAQKKWVHTTAVERKKIFLKFHDLVLKNRELLMDIVQLETGKNRASAADEVLDVAITTRFYANNAGKFLNDKKRPGALPIITKNTQQYVPKGVVGQITPWNYPLTLGVSDAVPALLAGNAVVAKPDLATPFSCLIMVHLLIEAGLPRDLMQVVTGPGDIVGGAIAAQCDFLMFTGSTATGRILGRTMGERLVGFSAELGGKNPLIVAKDADLDKVEAELPQACFSNSGQLCVSTERIYVEEDVYEEVIARFSKAAKAMSIGAGFEWKYEMGSLINQAQLDRVSTFVDQAKAAGATVLCGGKSRPDIGPFFYEPTVLADVPEGTPLLTEEVFGPVVFIEKVATLEEAVDKANGTPYGLNASVFGSSETGNLVAGQLEAGGIGINDGYAATWASVSTPLGGMKQSGLGHRHGAEGITKYAEIRNIAEQRWMSMRGPAKMPRKVYSDTVATALKLGKIFKVLP

SEQ ID NO: 7 - DNA, 1473 bp, Corynebacterium glutamicum
atgactattaatgtctccgaactacttgccaaagtccccacgggtctactgattggtgattcctgggtggaagcatccgacggcggtactttcgatgtggaaaacccagcgacgggtgaaacaatcgcaacgctcgcgtctgctacttccgaggatgcactggctgctcttgatgctgcatgcgctgttcaggccgagtgggctaggatgccagcgcgcgagcgttctaatattttacgccgcggttttgagctcgtagcagaacgtgcagaagagttcgccaccctcatgaccttggaaatgggcaagcctttggctgaagctcgcggcgaagtcacctacggcaacgaattcctgcgctggttctctgaggaagcagttcgtctgtatggccgttacggaaccacaccagaaggcaacttgcggatgctgaccgccctcaagccagttggcccgtgcctcctgatcaccccatggaacttcccactagcaatggctacccgcaaggtcgcacctgcgatcgctgcaggttgtgtcatggtgctcaagccagctcgacttaccccgctgacctcccagtattttgctcagaccatgcttgatgccggtcttccagcaggtgtcctcaatgtggtctccggtgcttccgcctctgcgatttccaacccgattatggaagacgatcgccttcgtaaagtctccttcaccggctccaccccagttggccagcagctgctcaaaaaggctgccgataaagttctgcgcacctccatggaacttggtggcaacgcacctttcattgtcttcgaggacgccgacctagatctcgcgatcgaaggtgccatgggtgccaaaatgcgcaacatcggcgaagcttgcaccgcagccaaccgtttcttagtccacgaatccgtcgccgatgaattcggccgtcgcttcgctgcccgccttgaagagcaagtcctaggcaacggcctcgacgaaggcgtcaccgtgggccccctggttgaggaaaaagcacgagacagcgttgcatcgcttgtcgacgccgccgtcgccgaaggtgccaccgtcctcaccggcggcaaggccggcacaggtgcaggctacttctacgaaccaacggtgctcacgggagtttcaacagatgcggctatcctgaacgaagagatcttcggtcccgtcgcaccgatcgtcaccttccaaaccgaggaagaagccctgcgtctagccaactccaccgaatacggactggcctcctatgtgttcacccaggacacctcacgtattttccgcgtctccgatggtctcgagttcggcctagtgggcgtcaattccggtgtcatctctaacgctgctgcaccttttggtggcgtaaaacaatccggaatgggccgcgaaggtggtctcgaaggaatcgaggagtacacctccgtgcagtacatcggtatccgggatccttacgccggctag

SEQ ID NO: 8 - DNA, 1362 bp, Corynebacterium glutamicum
gtgtctttgaccttcccagtaatcaaccccagcgatggctccaccatcaccgagctagaaaaccacgattccacccagtggatgtccgcgctctctgatgcagttgcagctggtccttcatgggctgcgaaaactccccgcgaaagatccgtggtactcaccgcaatcttcgaagcactgaccgaacgcgcccaagaacttgcagagatcatccacctggaagctggaaaatccgttgcagaagctcttggtgaagtcgcttatggtgcagaatacttccgttggtttgcggaagaagcagtgcgcctgcccggccgctacggacagtcaccttccggaatcggtcacatcgccgtcacccgcgcacccgtgggaccagtgctggcgatcaccccatggaatttccccatcgccatggccacccgcaaaatcgccccagccctggccgctggttgccccgtgttggtgaaacctgcttccgaaaccccactgaccatggtcaaagtgggggagatcatcgcctccgtctttgatacctttaatatcccgcagggcttggtctcaatcatcaccaccactcgagatgcagagctatcggcagaactcatggctgatcctcgcttggctaaagtcaccttcactggatcaaccaacgtgggacgcatcctggtccgccaatccgcggaccgactgctgcgcacctccatggaactcggcggaaatgcagcttttgttatcgacgaagccgcagacctcgacgaagccgtatccggtgccatcgccgcaaaactccgcaacgccggccaagtatgcatcgcagctaaccgtttcttggttcatgaatcccgcgctgccgaattcacctcaaagctggcgacagccatgcagaacactcccattgggccggtgatttctgcccgccaacgcgaccggatcgcagcactagtggatgaagccatcaccgacggcgcccgcctcatcatcggtggggaggtccccgacggctccggcttcttctatccagccaccatcttggccgatgtccctgcacagtcacggattgtgcatgaggaaatcttcggacctgtggccaccattgccactttcaccgacttggccgaaggcgttgcacaagcaaattccaccgaattcggcctcgcagcctacggattcagcaacaatgtgaaagcaacacagtacatggcggaacacttggaagccggaatggtcggaatcaacagaggcgccatctctgacccagcagcaccttttggcggcatcggacaatccggcttcggcagagaaggcggaaccgaaggaatcgaagaatatctctccgtgcgttacctcgctttgccgtga

SEQ ID NO: 9 - DNA, 1566 bp, Corynebacterium glutamicum
atgatcaaacgtcttcctttaggtccgctgcctaaagaacttcatcagactctgcttgatctgaccgcaaatgcccaagatgcggcgaaagtggaggttatagcgccatttactggcgagaccctcggatttgtttttgatggtgatgagcaagacgtcgagcatgcttttgcactttcaagggcagcccagaaaaagtgggtgcacaccacggcagtggaacggaagaagatcttcctgaagtttcatgatctggtattgaaaaaccgtgagctgctcatggacatcgtgcagttggaaacaggcaaaaatcgagcatcggctgccgatgaggtgttggacgttgcgatcaccacccgcttctacgcaaacaatgcaggaaagtttttaaatgacaagaaacgccccggcgcgcttccgatcatcacgaaaaacacacaacagtatgtgcccaagggagtggtcgggcagatcacgccgtggaattaccctttaactttgggagtatctgatgctgttccggcgctgctggcaggaaacgcagtggtggctaaacctgacctcgcgacacctttctcctgcttgatcatggtgcacctgctcattgaagccggtctgccgcgtgatttgatgcaggttgtcaccggccctggcgatattgttggcggtgcgattgcagctcagtgtgatttcctcatgttcactggatccacggccacgggccggatcttgggtcggacaatgggtgagcgtttggtgggtttctctgcggaattaggcggaaagaaccctcttattgtggccaaggatgcagatctggacaaggtggaagctgagcttccgcaggcgtgtttttccaactcggggcaattgtgtgtctccactgaacgtatttatgtcgaggaagacgtgtacgaggaggtgattgcacggtttagcaaggcggcgaaagccatgtccattggtgccggatttgagtggaaatatgagatgggttcgttgatcaatcaggcgcagctggatcgggtgagcacctttgttgatcaggctaaagctgcgggcgccacggtgctgtgcggtggcaagtcacgccctgatattggtcccttcttctatgagcccacggtattggcggatgtcccagagggcaccccactgctcacggaggaagtcttcgggccggtggtgttcatcgaaaaggtagccacactggaagaagccgtcgataaggcaaatggcacgccctacggcctgaatgcgtccgtctttgggtcgtcggaaaccggcaatcttgttgcaggccagctggaagctggcggtatcggtattaatgatggctacgccgcgacgtgggcgagcgtgtccacgcctctgggtggcatgaagcagtcggggctggggcaccgccatggtgcggagggaattacaaaatatgcggagatccgaaacatcgcggagcagcgctggatgtctatgcgtgggccggccaaaatgccgcgaaaggtgtactcagacaccgtggccacagcgctaaagctgggcaaaatctttaaagttttgccgtag

SEQ ID NO: 10 - protein, 371 aa, Porphyromonas gingivalis
MQLFKLKSVTHHFDTFAEFAKEFCLGERDLVITNEFIYEPYMKACQLPCHFVMQEKYGQGEPSDEMMNNILADIRNIQFDRVIGIGGGTVIDISKLFVLKGLNDVLDAFDRKIPLIKEKELIIVPTTCGTGSEVTNISIAEIKSRHTKMGLADDAIVADHAIIIPELLKSLPFHFYACSAIDALIHAIESYVSPKASPYSRLFSEAAWDIILEVFKKIAEHGPEYRFEKLGEMIMASNYAGIAFGNAGVGAVHALSYPLGGNYHVPHGEANYQFFTEVFKVYQKKNPFGYIVELNWKLSKILNCQPEYVYPKLDELLGCLLTKKPLHEYGMKDEEVRGFAESVLKTQQRLLANNYVELTVDEIEGIYRRLY

SEQ ID NO: 11 - DNA, 1116 bp, Porphyromonas gingivalis
atgcagcttttcaagctcaagagcgtcacacatcactttgatacttttgcagagtttgccaaggagttctgtctcggtgaacgcgacttggtaattaccaacgagttcatctacgaaccgtatatgaaggcatgccagctgccttgtcattttgtgatgcaggagaaatacggccaaggcgagccttctgacgagatgatgaacaacatcctagcagatatccgtaatatccagttcgaccgcgtgatcgggatcggaggtggtacggttattgacatctcaaaactctttgttctgaagggattaaatgatgttctcgacgcgttcgatcgcaagattccccttatcaaagagaaagaactgatcattgtgcccaccacctgcggaaccggctcggaggtgacgaacatttccatcgccgagatcaagtcccggcacaccaagatgggtttggctgacgatgcaattgttgctgaccacgccataatcatccctgaacttctgaagagcttgcccttccacttctatgcatgctccgcaatcgacgctcttattcatgccatcgagtcatacgtttctccaaaagcgtctccatactcccgtctgttcagtgaggcggcgtgggacattatcctggaagttttcaagaaaatcgccgaacacggcccagagtaccgcttcgagaagctgggggaaatgatcatggccagcaactatgccggtatcgctttcggcaacgcaggcgttggcgccgtccacgctctatcctacccgttgggcggcaactatcacgtgccgcatggagaagcaaactatcagttcttcaccgaggtctttaaagtataccaaaagaagaatccgttcggctatattgtcgaactcaactggaagctctccaagattctgaactgccagccagagtacgtgtacccgaagctggatgaactgctcggttgccttcttaccaagaaacctttgcacgaatacggcatgaaggacgaagaggttcgtggcttcgcggaatcggtcctgaagacccagcaacgcttgctcgccaacaactacgtcgaacttactgtcgatgagatcgaaggtatctaccgacgtctctactag

SEQ ID NO: 12 - protein, 538 aa, Clostridium kluyveri
MSKGIKNSQLKKKNVKASNVAEKIEEKVEKTDKVVEKAAEVTEKRIRNLKLQEKVVTADVAADMIENGMIVAISGFTPSGYPKEVPKALTKKVNALEEEFKVTLYTGSSTGADIDGEWAKAGIIERRIPYQTNSDMRKKINDGSIKYADMHLSHMAQYINYSVIPKVDIAIIEAVAITEEGDIIPSTGIGNTATFVENADKVIVEINEAQPLELEGMADIYTLKNPPRREPIPIVNAGNRIGTTYVTCGSEKICAIVMTNTQDKTRPLTEVSPVSQAISDNLIGFLNKEVEEGKLPKNLLPIQSGVGSVANAVLAGLCESNFKNLSCYTEVIQDSMLKLIKCGKADVVSGTSISPSPEMLPEFIKDINFFREKIVLRPQEISNNPEIARRIGVISINTALEVDIYGNVNSTHVMGSKMMNGIGGSGDFARNAYLTIFTTESIAKKGDISSIVPMVSHVDHTEHDVMVIVTEQGVADLRGLSPREKAVAIIENCVHPDYKDMLMEYFEEACKSSGGNTPHNLEKALSWHTKFIKTGSMK

SEQ ID NO: 13 - DNA, 1617 bp, Clostridium kluyveri
atgtctaaaggaatcaagaatagccaattgaaaaaaaagaacgtcaaggccagtaacgttgctgagaagatcgaagagaaggtggaaaagaccgacaaggtcgttgagaaggctgctgaggtgaccgaaaagcgcattcgaaacttaaagctccaggaaaaagttgtgaccgcagatgtcgcagctgacatgatcgagaatggcatgatcgtcgcaattagcggcttcacgccatccgggtatccaaaggaggttccaaaagcccttactaagaaggttaatgcgctggaggaggagttcaaggtgacgctgtataccggttctagcacaggcgctgatattgacggagaatgggcgaaggcaggaataatcgaacggcgtatcccataccagaccaactctgacatgaggaaaaaaataaacgatggttcaatcaagtacgcagatatgcacctgagccacatggctcaatacattaactattctgtgattcctaaggttgacattgccatcatcgaggcggtggccattaccgaggaaggggatattattcctagtactggaatcggcaacacagctacgtttgtcgagaatgcggataaggtaattgtggaaataaacgaggctcagccgcttgagttggaaggcatggcagatatctataccctgaagaaccctccacgtcgcgagcccatcccgatagtcaacgcaggcaaccgcatagggaccacttacgtcacctgtggctctgaaaaaatctgcgcgatcgtcatgaccaacacccaagacaaaacccgcccactcaccgaagtttctcctgtcagtcaggcaatctccgataacctgattggcttcctgaacaaagaagtagaggagggtaaactcccaaaaaacctgctccccatacagtcaggtgtcggttcggttgctaacgccgttctagccggactctgcgaatcaaacttcaaaaatttgagctgctacacagaagtgatccaggattcgatgttgaagctcatcaaatgtggaaaggcagatgtggtgtccggcacctcgatctcgccatcaccggaaatgctgcccgagttcataaaggacataaatttttttcgcgagaagatagtactgcgcccccaggaaatatctaataatccggaaatagctcgtcgtataggagtgatctccataaacactgctttggaagtagacatctacggtaatgtgaactccacgcatgtcatgggctccaagatgatgaacggcatcggcggcagcggcgactttgcccgcaacgcatacctcaccatattcactacggagtccatcgcgaagaagggcgacatttcctctatcgttcctatggtttcccacgtggaccacaccgagcatgacgtaatggtcatcgttaccgaacagggggttgcggatctgcgcggtctttcccctcgggaaaaggccgtggcgataattgagaattgcgtccacccggattacaaggatatgctcatggagtacttcgaggaggcttgtaagtcctcaggtggcaacaccccacacaaccttgaaaaagccctatcctggcacactaagttcataaaaactggctcgatgaagtaa

SEQ ID NO: 14 - protein, 431 aa, Porphyromonas gingivalis
MKDVLAEYASRIVSAEEAVKHIKNGERVALSHAAGVPQSCVDALVQQADLFQNVEIYHMLCLGEGKYMAPEMAPHFRHITNFVGGNSRKAVEENRADFIPVFFYEVPSMIRKDILHIDVAIVQLSMPDENGYCSFGVSCDYSKPAAESAHLVIGEINRQMPYVHGDNLIHISKLDYIVMADYPIYSLAKPKIGEVEEAIGRNCAELIEDGATLQLGIGAIPDAALLFLKDKKDLGIHTEMFSDGVVELVRSGVITGKKKTLHPGKMVATFLMGSEDVYHFIDKNPDVELYPVDYVNDPRVIAQNDNMVSINSCIEIDLMGQVVSECIGSKQFSGTGGQVDYVRGAAWSKNGKSIMAIPSTAKNGTASRIVPIIAEGAAVTTLRNEVDYVVTEYGIAQLKGKSLRQRAEALIAIAHPDFREELTKHLRKRFG

SEQ ID NO: 15 - DNA, 1296 bp, Porphyromonas gingivalis
atgaaggatgtactggcggaatacgcctcccgcattgtttcggcggaggaggccgttaagcacatcaaaaacggtgaacgggtagctttgtcacacgctgccggcgtgcctcagagttgcgttgacgcactggtgcagcaggccgaccttttccagaatgtggaaatctatcacatgctgtgcctcggtgagggtaagtatatggcgcctgagatggcccctcacttccgccacatcaccaactttgtcggtggtaactcccgtaaggcggtcgaagaaaaccgggccgatttcattccggtattcttttacgaggtgccaagcatgattcgcaaagacatcctccacattgatgtcgccatcgttcagctttcaatgcctgacgaaaatggttactgttcctttggagtatcttgcgattactccaagccggcagcagagagcgctcacctggttatcggagaaatcaaccgtcaaatgccatacgtacacggcgacaacttgattcatatctccaagttggattacatcgtgatggcagactaccccatctactctcttgcaaagcccaagatcggggaagtcgaggaagctatcgggaggaattgtgccgagcttattgaagatggtgccactctccagctgggaatcggcgcgattcctgatgcggccctgttatttctcaaggacaaaaaggatctgggcatccataccgaaatgttctccgatggtgttgtcgaattggttcgctccggcgttatcacaggcaagaaaaagactcttcaccccggaaagatggtcgcaaccttcctgatgggaagcgaggacgtgtatcatttcatcgataaaaaccccgatgtagaactgtatccagtagattacgtgaatgacccgcgtgtgatcgcccaaaacgacaatatggtctcgattaacagctgcatcgaaatcgaccttatgggacaggtcgtgtccgagtgcatcggctcaaagcaattcagcggcaccggcggccaagttgactacgtgcgtggcgcagcatggtctaaaaacggcaaatcgatcatggcaatcccgtccactgcaaaaaacggtacggcatctcgaattgtacctatcatcgcggagggcgctgctgtcaccaccctgcgcaacgaggtcgattacgttgtaaccgagtacggtatcgctcagctcaagggcaagagcctgcgccagcgcgcagaggctttgatcgcgatagcccaccccgacttccgtgaggaactaacgaaacatctccgcaagcgattcggataa

SEQ ID NO: 16 - protein, 858 aa, Clostridium acetobutylicum
MKVTNQKELKQKLNELREAQKKFATYTQEQVDKIFKQCAIAAAKERINLAKLAVEETGIGLVEDKIIKNHFAAEYIYNKYKNEKTCGIIDHDDSLGITKVAEPIGIVAAIVPTTNPTSTAIFKSLISLKTRNAIFFSPHPRAKKSTIAAAKLILDAAVKAGAPKNIIGWIDEPSIELSQDLMSEADIILATGGPSMVKAAYSSGKPAIGVGAGNTPAIIDESADIDMAVSSIILSKTYDNGVICASEQSILVMNSIYEKVKEEFVKRGSYILNQNEIAKIKETMFKNGAINADIVGKSAYIIAKMAGIEVPQTTKILIGEVQSVEKSELFSHEKLSPVLAMYKVKDFDEALKKAQRLIELGGSGHTSSLYIDSQNNKDKVKEFGLAMKTSRTFINMPSSQGASGDLYNFAIAPSFTLGCGTWGGNSVSQNVEPKHLLNIKSVAERRENMLWFKVPQKIYFKYGCLRFALKELKDMNKKRAFIVTDKDLFKLGYVNKITKVLDEIDIKYSIFTDIKSDPTIDSVKKGAKEMLNFEPDTIISIGGGSPMDAAKVMHLLYEYPEAEIENLAINFMDIRKRICNFPKLGTKAISVAIPTTAGTGSEATPFAVITNDETGMKYPLTSYELTPNMAIIDTELMLNMPRKLTAATGIDALVHAIEAYVSVMATDYTDELALRAIKMIFKYLPRAYKNGTNDIEAREKMAHASNIAGMAFANAFLGVCHSMAHKLGAMHHVPHGIACAVLIEEVIKYNATDCPTKQTAFPQYKSPNAKRKYAEIAEYLNLKGTSDTEKVTALIEAISKLKIDLSIPQNISAAGINKKDFYNTLDKMSELAFDDQCTTANPRYPLISELKDIYIKSF

SEQ ID NO: 17 - DNA, 2577 bp, Clostridium acetobutylicum
atgaaagtaaccaatcagaaagagttgaagcagaagttgaacgagctgcgagaggctcagaagaagttcgcaacctacacccaggaacaggtggacaagatctttaagcagtgtgccattgcagccgcgaaagaacgtattaatctcgcgaaacttgcggtcgaggaaaccggtattgggctggtagaagacaagatcatcaagaaccacttcgccgctgaatacatctacaacaagtacaaaaacgaaaagacatgtggtatcatcgaccacgacgacagcttgggcatcaccaaggtagcggagccaatcggtatcgtcgcagctatcgtgcccactactaaccctacctccactgctattttcaagtcactcatctccctgaaaacccgcaatgctatcttcttctcacctcacccacgcgctaagaaatcaactatcgctgcagctaaacttatcctggatgcagccgtgaaagccggggctccgaaaaacatcatcggttggatcgacgaaccttccattgaactctctcaagacctcatgtccgaggcagacattatcctggcaaccggaggcccatccatggttaaagcagcttacagctcaggcaagccggctatcggcgttggagctggtaacactccagcaatcatcgacgagtcggccgatatcgacatggcagtgtcctctattatcctgtccaaaacttatgacaacggcgttatttgcgcgtccgagcagtctattctcgtcatgaactctatttacgagaaggtaaaggaggagtttgtgaagcgggggtcgtacattctgaaccagaacgagatcgctaagatcaaagagactatgtttaaaaacggagccatcaacgcagatatcgtagggaagtccgcgtacatcattgctaagatggctggaatcgaagtccctcaaaccacgaaaattctgatcggcgaggtgcaatcggtcgaaaagtccgagctgttctcgcatgaaaagttgtccccggtcctcgcgatgtataaagttaaggattttgatgaagcactcaagaaagctcagcgcctgatcgaattgggtggctcgggtcacacctcttccctctacattgactcccagaacaataaagataaggtgaaagagttcggcctggctatgaagacgtctcgtaccttcatcaatatgccctcttcacagggcgccagcggtgacctttacaatttcgctatcgctcctagctttaccctcggctgcggcacctggggcggtaattctgtgtcccaaaacgtcgaaccaaagcatctgctcaacattaaaagcgtcgccgaacgtcgcgagaacatgttgtggttcaaggtcccgcaaaaaatctacttcaagtatggttgcttgcgctttgcacttaaagagcttaaggacatgaataaaaagcgggcgttcatcgtcactgataaggatctgttcaaactgggctatgttaacaagattaccaaggtcctggatgagatcgatatcaagtattccatcttcaccgatattaagtccgatccgaccattgattccgtgaagaagggcgcgaaggagatgctcaactttgaacccgacacgattatttctattggcggaggcagcccaatggacgcagctaaggttatgcacctgctgtatgagtacccagaagcagagatcgagaaccttgcaatcaatttcatggatattcgcaaacgcatttgcaactttcctaagcttggtacaaaagctatctctgttgcgatccctaccaccgcaggaaccggcagcgaagcgacaccattcgccgttattaccaacgatgaaacaggtatgaagtacccacttacctcttatgaacttaccccgaacatggctatcattgatacggaattgatgctgaacatgccgcggaagttgaccgcagctacgggaatcgatgcattggttcatgcaatcgaggcatacgtttccgtcatggcaaccgattacaccgacgagctcgcgttgcgtgcgattaaaatgatcttcaagtaccttccacgcgcatacaagaatggcacaaacgatattgaagcccgagaaaagatggcacacgcttcgaacatcgctggtatggccttcgcgaatgcgtttctcggagtgtgtcactccatggcgcacaaactgggagccatgcatcacgtgccccacggtatcgcatgcgccgttcttattgaagaggtgatcaagtataatgccaccgattgccccactaagcagacggccttccctcagtacaaatcgcccaatgccaagcgtaaatacgcggaaattgccgagtacttgaaccttaaggggaccagcgacacggaaaaggtgaccgcactgattgaagccatctccaagcttaagatcgacctgagcatcccacaaaacatctcagcagccggcattaacaagaaggacttctacaacactctcgacaagatgtcagagctcgccttcgatgatcagtgcactaccgcaaacccacgttatccgctcatctctgaactgaaggatatctacatcaagtcgttttaa

SEQ ID NO: 18 - DNA, 43 bp, Artificial Sequence
  Synthetic (ldhA_5′_HindIII)
catgattacgccaagcttgagagcccaccacattgcgatttcc

SEQ ID NO: 19 - DNA, 42 bp, Artificial Sequence
  Synthetic (ldhA_up_3′_XhoI)
tcgaaactcgagtttcgatcccacttcctgatttccctaacc

SEQ ID NO: 20 - DNA, 39 bp, Artificial Sequence
  Synthetic (ldhA_dn_5′_XhoI)
tcgaaactcgagtaaatctttggcgcctagttggcgacg

SEQ ID NO: 21 - DNA, 46 bp, Artificial Sequence
  Synthetic (ldhA_3′_EcoRI)
acgacggccagtgaattcgacgacatctgagggtggataaagtggg

SEQ ID NO: 22 - DNA, 20 bp, Artificial Sequence
  Synthetic (ldhA up)
atcgggcataattaaaggtg

SEQ ID NO: 23 - DNA, 22 bp, Artificial Sequence
  Synthetic (ldhA down)
gtcacctcatcaagttctagaa

SEQ ID NO: 24 - DNA, 36 bp, Artificial Sequence
  Synthetic (0049-1 for)
gcaggcatgcaagcttaaagtccccacgggtctact

SEQ ID NO: 25 - DNA, 35 bp, Artificial Sequence
  Synthetic (0049-1 rev)
gagctcagtcagtcacggagaccacattgaggaca

SEQ ID NO: 26 - DNA, 35 bp, Artificial Sequence
  Synthetic (0049-2 for)
tgactgactgagctcccaaaatgcgcaacatcggc

SEQ ID NO: 27 - DNA, 36 bp, Artificial Sequence
  Synthetic (0049-2 rev)
ggccagtgccaagctttaccgatgtactgcacggag

SEQ ID NO: 28 - DNA, 36 bp, Artificial Sequence
  Synthetic (0463-1 for)
gcaggcatgcaagcttgcaccagaaggtgggctaaa

SEQ ID NO: 29 - DNA, 35 bp, Artificial Sequence
  Synthetic (0463-1 rev)
gagctcagtcagtcatgccgatagctctgcatctc

SEQ ID NO: 30 - DNA, 35 bp, Artificial Sequence
  Synthetic (0463-2 for)
tgactgactgagctcgtatgcatcgcagctaaccg

SEQ ID NO: 31 - DNA, 36 bp, Artificial Sequence
  Synthetic (0463-2 rev)
ggccagtgccaagctttacctgcagcattgtgcgca

SEQ ID NO: 32 - DNA, 36 bp, Artificial Sequence
  Synthetic (2619-1 for)
gcaggcatgcaagcttgtcttcctttaggtccgctg

SEQ ID NO: 33 - DNA, 35 bp, Artificial Sequence
  Synthetic (2619-1 rev)
gagctcagtcagtcaccggtgacaacctgcatcaa

SEQ ID NO: 34 - DNA, 35 bp, Artificial Sequence
  Synthetic (2619-2 for)
tgactgactgagctcggtgccggatttgagtggaa

SEQ ID NO: 35 - DNA, 36 bp, Artificial Sequence
  Synthetic (2619-2 rev)
ggccagtgccaagcttttgcccagctttagcgctgt

SEQ ID NO: 36 - DNA, 20 bp, Artificial Sequence
  Synthetic (0049for)
gtgatgctggtaaacctgtg

SEQ ID NO: 37 - DNA, 19 bp, Artificial Sequence
  Synthetic (0049rev)
ttgcgtttgggaaatacgg

SEQ ID NO: 38 - DNA, 20 bp, Artificial Sequence
  Synthetic (0463for)
gcaccagaaggtgggctaaa

SEQ ID NO: 39 - DNA, 20 bp, Artificial Sequence
  Synthetic (0463rev)
tacctgcagcattgtgcgca

SEQ ID NO: 40 - DNA, 20 bp, Artificial Sequence
  Synthetic (2619for)
gtcttcctttaggtccgctg

SEQ ID NO: 41 - DNA, 20 bp, Artificial Sequence
  Synthetic (2619rev)
ttgcccagctttagcgctgt

SEQ ID NO: 42 - DNA, 4990 bp, Artificial Sequence
  Synthetic (pGSK+)
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgcccctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccgggccccccctcgaggtcgacggtatcgataagcttgatatcgaattcctgcagcccgggggatccactagttctagagcggccgccaccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctgggaacaacaagacccatcatagtttgcccccgcgacattgaccataaattcatcgcacaaaatatcgaacggggtttatgccgcttttagtgggtgcgaagaatagtctgctcattacccgcgaacaccgccgcattcagatcacgcttagtagcgtccccatgagtaggcagaaccgcgtccaagtccacatcatccataacgatcatgcacggggtggaatccacacccagacttgccagcacctcattagcgacacgttgcgcagcggccacgtccttagccttatccacgcaatctaggacgtactgcctaaccgcgaaatcagactgaatcagtttccaatcatcgggcttcaccaaagcaacagcaacgcgggttgattcgacccgttccggtgcttccagaccggcgagcttgtacagttcttcttccatttcacgacgtacatcagcgtctatgtaatcaatgcccaaagcacgcttagccccacgtgaccaggacgaacgcaggtttttagaaccaacctcatactcacgccaccgagccaccaaaacagcgtccatatcctcgccggcgtcgctttgatcggccaacatatccaacatctgaaacggcgtgtacgaccccttagacgcggttttagtagcggagccagtcagttcctgagacatgcccttagcgaggtaggttgccattttcgcagcgtctccaccccaggtagacacctgatcaagtttgaccccgtgctcacgcagtggcgcgtccataccggccttaaccacaccagcagaccagcgggaaaacatggaatcctcaaacgccttgagttcatcgtcagacagtggacgatccaagaacaacagcatgttgcggtgcaagtgccaaccgttcgcccaagagtctgtgacctcatagtcactataggtgtgctccaccccgtaccgtgcacgttctttcttccactgagatgttttcaccatcgaagagtacgcagtcttaatacccgcttcaacctgcgcaaatgactgtgagcggttgtgtcgaacagtgcccacaaacatcatgagcgcgccacccgccgccaagtgattcttagtagcaatagccagctcaatgcggcgttcgcccatgacttccaattcagccagaggtgacccccagcgagagtgagagttttgcagaccctcaaactgcgaagcaccgttagacgaccaggacaccgcaacagcttcgtccctgcgccacctatggcaccccgccagagccttactattggtgatcttgtacatgacgttttgcctacgccacgccctagcgcgagtgaccttagaaccctcattgacctgcggttccttagaggtgttcacttctatttcagtgttacctagacccgatgttgtgcggggttgcgcagtgcgagtttgtgcgggtgttgtgcccgttgtcttagctagtgctatggttgtcaattgaaaccccttcgggttatgtggcccccgtgcatatgagttggtagctcgcacgggggtttgtcttgtctaggaactattaatttttagtggtgtttggtggccgcctagcttggctatgcgtgccagcttacccgtactcaatgttaaagatttgcatcgacatgggagggttacgtgtccgatacctagggggggtatccgcgactaggtgccccggtgctcactgtctgtaccggcggggcaagccccacaccccgcatggacagggtggctccgccccctgcacccccagcaatctgcatgtacatgttttacacattagcacgacatgactgcatgtgcatgcactgcatgcagactaggtaaatatgagtatgtacgactagtaacaggagcactgcacataatgaatgagttgcaggacaatgtttgctacgcatgcgcatgacatatcgcaggaaagctactagagtcttaaagcatggcaaccaaggcacagctagaacagcaactacaagaagctcaacaggcactacaggcgcagcaagcgcaggcacaagccaccatcgaagcactagaagcgcaggcaaaggctaagcccgtcgtggtcaccgcacgcgttcctttggcactacgtgaggacatgaagcgcgcaggcatgcagaacggtgaaaacctccaagagttcatgatcgccgcgtttaccgagcggctagaaaagctcaccaccaccgacaacgaggaaaacaatgtctaacccactagttctctttgcccaccgtgacccggtaaatgacgtgacgttcgagtgcattgagcacgccacctacgacacactttcacacgctaaagaccagatcaccgcccaaatgcaagccctagacgaagaagccgccctactgccctaatgggtgtttcatgggtgtttccctagtgtttcatggtgttttcacctaagctagggaattgcgcgagaagtctcgcaaaaatcagcaacccccggaaccacacagttcacgggggttcttctatgccagaaatcagaaaggggaaccagtgaacgaccccgaatattggatcacagcgcagcaggtcgccgcccgcgtagctctcaccccggccaccattaaaaagtgggcaaacgagggaaaaatcaccgcatacaagatcggcaagtccgtccgattcaaagcatcagacgtagacaagctaggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcataacaccccttgtattactgtttatgtaagcagacagttttattgttcatgatgatatatttttatcttgtgcaatgtaacatcagagattttgagacacaacgtggctttccccccccccccaaaaggatctaggtgaagatcctttttgataatctcatgaccaaaatcccttaacgtgagttttcgttccactgagcgtcagaccccgtagaaaagatcaaaggatcttc

SEQ ID NO: 43 - DNA, 5342 bp, Artificial Sequence
  Synthetic (pGT1)
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgcccctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccgggccccccctcgaggtcgacggtatcgataagcttgatatcgaattcctgcagcccgggggatccactagttctagagcggccgccaccgcggtggagctcatttagcggatgattctcgttcaacttcggccgaagccacttcgtctgtcataatgacagggatggtttcggccgtttttgcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccatccccccacagatacggtagctccagcttttgttccctttagtgagggttaatttcgagcttggcgtaatcatggtcatagctgtttcctgtgtgaaattgttatccgctcacaattccacacaacatacgagccggaagcataaagtgtaaagcctgggaacaacaagacccatcatagtttgcccccgcgacattgaccataaattcatcgcacaaaatatcgaacggggtttatgccgcttttagtgggtgcgaagaatagtctgctcattacccgcgaacaccgccgcattcagatcacgcttagtagcgtccccatgagtaggcagaaccgcgtccaagtccacatcatccataacgatcatgcacggggtggaatccacacccagacttgccagcacctcattagcgacacgttgcgcagcggccacgtccttagccttatccacgcaatctaggacgtactgcctaaccgcgaaatcagactgaatcagtttccaatcatcgggcttcaccaaagcaacagcaacgcgggttgattcgacccgttccggtgcttccagaccggcgagcttgtacagttcttcttccatttcacgacgtacatcagcgtctatgtaatcaatgcccaaagcacgcttagccccacgtgaccaggacgaacgcaggtttttagaaccaacctcatactcacgccaccgagccaccaaaacagcgtccatatcctcgccggcgtcgctttgatcggccaacatatccaacatctgaaacggcgtgtacgaccccttagacgcggttttagtagcggagccagtcagttcctgagacatgcccttagcgaggtaggttgccattttcgcagcgtctccaccccaggtagacacctgatcaagtttgaccccgtgctcacgcagtggcgcgtccataccggccttaaccacaccagcagaccagcgggaaaacatggaatcctcaaacgccttgagttcatcgtcagacagtggacgatccaagaacaacagcatgttgcggtgcaagtgccaaccgttcgcccaagagtctgtgacctcatagtcactataggtgtgctccaccccgtaccgtgcacgttctttcttccactgagatgttttcaccatcgaagagtacgcagtcttaatacccgcttcaacctgcgcaaatgactgtgagcggttgtgtcgaacagtgcccacaaacatcatgagcgcgccacccgccgccaagtgattcttagtagcaatagccagctcaatgcggcgttcgcccatgacttccaattcagccagaggtgacccccagcgagagtgagagttttgcagaccctcaaactgcgaagcaccgttagacgaccaggacaccgcaacagcttcgtccctgcgccacctatggcaccccgccagagccttactattggtgatcttgtacatgacgttttgcctacgccacgccctagcgcgagtgaccttagaaccctcattgacctgcggttccttagaggtgttcacttctatttcagtgttacctagacccgatgttgtgcggggttgcgcagtgcgagtttgtgcgggtgttgtgcccgttgtcttagctagtgctatggttgtcaattgaaaccccttcgggttatgtggcccccgtgcatatgagttggtagctcgcacgggggtttgtcttgtctaggaactattaatttttagtggtgtttggtggccgcctagcttggctatgcgtgccagcttacccgtactcaatgttaaagatttgcatcgacatgggagggttacgtgtccgatacctagggggggtatccgcgactaggtgccccggtgctcactgtctgtaccggcggggcaagccccacaccccgcatggacagggtggctccgccccctgcacccccagcaatctgcatgtacatgttttacacattagcacgacatgactgcatgtgcatgcactgcatgcagactaggtaaatatgagtatgtacgactagtaacaggagcactgcacataatgaatgagttgcaggacaatgtttgctacgcatgcgcatgacatatcgcaggaaagctactagagtcttaaagcatggcaaccaaggcacagctagaacagcaactacaagaagctcaacaggcactacaggcgcagcaagcgcaggcacaagccaccatcgaagcactagaagcgcaggcaaaggctaagcccgtcgtggtcaccgcacgcgttcctttggcactacgtgaggacatgaagcgcgcaggcatgcagaacggtgaaaacctccaagagttcatgatcgccgcgtttaccgagcggctagaaaagctcaccaccaccgacaacgaggaaaacaatgtctaacccactagttctctttgcccaccgtgacccggtaaatgacgtgacgttcgagtgcattgagcacgccacctacgacacactttcacacgctaaagaccagatcaccgcccaaatgcaagccctagacgaagaagccgccctactgccctaatgggtgtttcatgggtgtttccctagtgtttcatggtgttttcacctaagctagggaattgcgcgagaagtctcgcaaaaatcagcaacccccggaaccacacagttcacgggggttcttctatgccagaaatcagaaaggggaaccagtgaacgaccccgaatattggatcacagcgcagcaggtcgccgcccgcgtagctctcaccccggccaccattaaaaagtgggcaaacgagggaaaaatcaccgcatacaagatcggcaagtccgtccgattcaaagcatcagacgtagacaagctaggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcataacaccccttgtattactgtttatgtaagcagacagttttattgttcatgatgatatatttttatcttgtgcaatgtaacatcagagattttgagacacaacgtggctttccccccccccccaaaaggatctaggtgaagatcctttttgataatctcatgaccaaaatcccttaacgtgagttttcgttccactgagcgtcagaccccgtagaaaagatcaaaggatcttc

SEQ ID NO: 44 - DNA, 5693 bp, Artificial Sequence
  Synthetic (synthetic 4G cassette)
gtcgacatgattttgcatctgctgcgaaatctttgtttccccgctaaagttgaggacaggttgacacggagttgactcgacgaattatccaatgtgagtaggtttggtgcgtgagttggaaaaattcgccatactcgcccttgggttctgtcagctcaagaattcttgagtgaccgatgctctgattgacctaactgcttgacacattgcatttcctacaatctttagaggagacacaacatgtctaaaggaatcaagaatagccaattgaaaaaaaagaacgtcaaggccagtaacgttgctgagaagatcgaagagaaggtggaaaagaccgacaaggtcgttgagaaggctgctgaggtgaccgaaaagcgaattcgaaacttaaagctccaggaaaaagttgtgaccgcagatgtcgcagctgacatgatcgagaatggcatgatcgtcgcaattagcggcttcacgccatccgggtatccaaaggaggttccaaaagcccttactaagaaggttaatgcgctggaggaggagttcaaggtgacgctgtataccggttctagcacaggcgctgatattgacggagaatgggcgaaggcaggaataatcgaacggcgtatcccataccagaccaactctgacatgaggaaaaaaataaacgatggttcaatcaagtacgcagatatgcacctgagccatatggctcaatacattaactattctgtgattcctaaggttgacattgccatcatcgaggcggtggccattaccgaggaaggggatattattcctagtactggaatcggcaacacagctacgtttgtcgagaatgcggataaggtaattgtggaaataaacgaggctcagccgcttgagttggaaggcatggcagatatctataccctgaagaaccctccacgtcgcgagcccatcccgatagtcaacgcaggcaaccgcatagggaccacttacgtcacctgtggctctgaaaaaatctgcgcgatcgtcatgaccaacacccaagacaaaacccgcccactcaccgaagtttctcctgtcagtcaggcaatctccgataacctgattggcttcctgaacaaagaagtagaggagggtaaactcccaaaaaacctgctccccatacagtcaggtgtcggttcggttgctaacgccgtgctagccggactctgcgaatcaaacttcaaaaatttgagctgctacacagaagtgatccaggattcgatgttgaagcttatcaaatgtggaaaggcagatgtggtgtccggcacctcgatctcgccatcaccggaaatgctgcccgagttcataaaggacataaatttttttcgcgagaagatagtactgcgcccccaggaaatatctaataatccggaaatagctcgtcgtataggagtgatctccataaacactgctttggaagtagacatctacggtaatgtgaactccacgcatgtcatgggctccaagatgatgaacggcatcggcggcagcggcgactttgcccgcaacgcatacctcaccatattcactacggagtccatcgcgaagaagggcgacatttcctctatcgttcctatggtttcccacgtggaccacaccgagcatgacgtaatggtcatcgttaccgaacagggggttgcggatctccgcggtctttcccctcgggaaaaggccgtggcgataattgagaattgcgtccacccggattacaaggatatgctcatggagtacttcgaggaggcttgtaagtcctcaggtggcaacaccccacacaaccttgaaaaagccctatcctggcacactaagttcataaaaactggctcgatgaagtaattagaggagacacaacatggagattaaagagatggtcagtcttgcgcgcaaagctcagaaggagtatcaggccacccataaccaagaagctgtggacaacatctgccgagctgcagcgaaggttatttacgaaaatgcagcaattctggcccgcgaggcagtggacgaaaccggcatgggtgtttacgagcacaaggtggccaagaatcaaggcaagtccaaaggtgtttggtacaacctgcataacaagaagtcgattggcatcctcaatatcgatgagcgtaccggcatgatcgagatcgcaaaacctatcggggttgtaggcgccgttacgccaaccaccaaccctatcgttactccgatgagcaacatcatctttgctcttaagacctgcaacgccatcattatcgccccacacccgcgctccaaaaagtgctctgcccacgcagttcggctgatcaaagaggctatcgctccgttcaacgtgcccgaaggtatggttcagatcatcgaggagcctagcatcgagaagacgcaggaattgatgggcgccgtagacgtggtcgttgctaccgggggcatgggcatggtcaagtctgcctactcctcagggaagccttctttcggtgtcggagccggcaatgttcaggtgatagtggacagcaacatcgatttcgaagcggctgcagaaaagatcatcaccggacgtgccttcgacaacggtatcatctgctcaggcgaacagtccatcatctacaacgaggctgacaaggaagcagttttcacagcattccgcaaccacggtgcgtacttttgcgacgaggccgagggagatcgggctcgtgcagcgatcttcgaaaatggagccatcgcgaaagatgttgtgggccagtccgttgcctttattgccaagaaggcgaacattaatatccccgagggtactcgtattctcgtggtcgaagctcgcggagtaggcgccgaagatgtcatctgtaaagaaaagatgtgtccagtcatgtgcgccctctcctacaagcacttcgaagagggggtagagatcgcaaggacgaacctcgcaaacgaaggcaatggccatacctgtgctatccactccaacaaccaagcacacatcatcttggcaggctcggagctgaccgtgtctcgcatcgtggtcaacgcgccaagtgctaccacagcaggcggtcacatccagaacggtcttgccgtcaccaatactctaggctgcggctcttggggtaacaactcgatctccgaaaacttcacttataaacacctgctcaacatttcacgcatcgccccgttgaactccagcattcatatcccagatgataaggaaatctgggaactctaattagaggagacacaacatgcagcttttcaagctcaagagcgtcacacatcactttgatacttttgcagagtttgccaaggaattctgtctcggtgaacgcgacttggtaattaccaacgagttcatctacgaaccgtatatgaaggcatgccagctgccttgtcattttgtgatgcaggagaaatacggccaaggcgagccttctgacgagatgatgaacaacatcctagcagatatccgtaatatccagttcgaccgcgtgatcgggatcggaggtggtacggttattgacatctcaaaactctttgttctgaagggattaaatgatgttctcgacgcgttcgatcgcaagattccccttatcaaagagaaagaactgatcattgtgcccaccacctgcggaaccggctcggaggtgacgaacatttccatcgccgagatcaagtcccggcacaccaagatgggtttggctgacgatgcaattgttgctgaccacgccataatcatccctgaacttctgaagagcttgcccttccacttctatgcatgctccgcaatcgatgctcttattcatgccatcgagtcatacgtttctccaaaagcgtctccatactcccgtctgttcagtgaggcggcgtgggacattatcctggaagttttcaagaaaatcgccgaacacggcccagagtaccgcttcgagaagctgggggaaatgatcatggccagcaactatgccggtatcgctttcggcaacgcaggcgttggcgccgtccacgctctatcctacccgttgggcggcaactatcacgtgccgcatggagaagcaaactatcagttcttcaccgaggtctttaaagtataccaaaagaagaatccgttcggctatattgtcgaactcaactggaagctctccaagattctgaactgccagccagagtacgtgtacccgaagctggatgaactgctcggttgccttcttaccaagaaacctttgcacgaatacggcatgaaggacgaagaggttcgtggcttcgcggaatcggtcctgaagacccagcaacgcttgctcgccaacaactacgtcgaacttactgtcgatgagatcgaaggtatctaccgacgtctctactaattagaggagacacaacatgaaggatgtactggcggaatacgcctcccgcattgtttcggcggaggaggccgttaagcacatcaaaaacggtgaacgggtagctttgtcacacgctgccggcgtgcctcagagttgcgttgacgcactggtgcagcaggccgaccttttccagaatgtggaaatctatcacatgctgtgcctcggtgagggtaagtatatggcgcctgagatggcccctcacttccgccacatcaccaactttgtcggtggtaactcccgtaaggcggtcgaagaaaaccgggccgatttcattccggtattcttttacgaggtgccaagcatgattcgcaaagacatcctccacattgatgtcgccatcgttcagctttcaatgcctgacgaaaatggttactgttcctttggagtatcttgcgattactccaagccggcagcagagagcgctcacctggttatcggagaaatcaaccgtcaaatgccatacgtacacggcgacaacttgattcatatctccaagttggattacatcgtgatggcagactaccccatctactctcttgcaaagcccaagatcggggaagtcgaggaagctatcgggaggaattgtgccgagcttattgaagatggtgccactctccagctgggaatcggcgcgattcctgatgcggccctgttatttctcaaggacaaaaaggatctgggcatccataccgaaatgttctccgatggtgttgtcgaattggttcgctccggcgttatcacaggcaagaaaaagactcttcaccccggaaagatggtcgcaaccttcctgatgggaagcgaggacgtgtatcatttcatcgataaaaaccccgatgtagaactgtatccagtagattacgtgaatgacccgcgtgtgatcgcccaaaacgacaatatggtctcgattaacagctgcatcgaaatcgaccttatgggacaggtcgtgtccgagtgcatcggctcaaagcaattcagcggcaccggcggccaagttgactacgtgcgtggcgcagcatggtctaaaaacggcaaatcgatcatggcaatcccgtccactgcaaaaaacggtacggcatctcgaattgtacctatcatcgcggagggcgctgctgtcaccaccctgcgcaacgaggtcgattacgttgtaaccgagtacggtatcgctcagctcaagggcaagagcctgcgccagcgcgcagaggctttgatcgcgatagcccaccccgacttccgtgaggaactaacgaaacatctccgcaagcgattcggataagcggccgcagatctggatcc

SEQ ID NO: 45 - DNA, 44 bp, Artificial Sequence
  Synthetic (3G up)
gatatcgaattcctgcagatgattttgcatctgctgcgaaatct

SEQ ID NO: 46 - DNA, 47 bp, Artificial Sequence
  Synthetic (3G down)
gcggtggcggccgctctagattagtagagacgtcggtagataccttc

SEQ ID NO: 47 - DNA, 5567 bp, Artificial Sequence
  Synthetic (pGSX1)
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgcccctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccatgattttgcatctgctgcgaaatctttgtttccccgctaaagttgaggacaggttgacacggagttgactcgacgaattatccaatgtgagtaggtttggtgcgtgagttggaaaaattcgccatactcgcccttgggttctgtcagctcaagaattcttgagtgaccgatgctctgattgacctaactgcttgacacattgcatttcctacaatctttagaggagacacaacctcgaggtcgacggtatcgataagcttgatatcgaattcctgcagcccgggggatccactagttctagagcggccgccaccgcggtggagctcatttagcggatgattctcgttcaacttcggccgaagccacttcgtctgtcataatgacagggatggtttcggccgtttttgcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccatccccccacagatacggtagctccagcttttgttccctttagtgagggttaatttcgagcttggcgtaatcatggtcatagctgtttcctgtgtgaaattgttatccgctcacaattccacacaacatacgagccggaagcataaagtgtaaagcctgggaacaacaagacccatcatagtttgcccccgcgacattgaccataaattcatcgcacaaaatatcgaacggggtttatgccgcttttagtgggtgcgaagaatagtctgctcattacccgcgaacaccgccgcattcagatcacgcttagtagcgtccccatgagtaggcagaaccgcgtccaagtccacatcatccataacgatcatgcacggggtggaatccacacccagacttgccagcacctcattagcgacacgttgcgcagcggccacgtccttagccttatccacgcaatctaggacgtactgcctaaccgcgaaatcagactgaatcagtttccaatcatcgggcttcaccaaagcaacagcaacgcgggttgattcgacccgttccggtgcttccagaccggcgagcttgtacagttcttcttccatttcacgacgtacatcagcgtctatgtaatcaatgcccaaagcacgcttagccccacgtgaccaggacgaacgcaggtttttagaaccaacctcatactcacgccaccgagccaccaaaacagcgtccatatcctcgccggcgtcgctttgatcggccaacatatccaacatctgaaacggcgtgtacgaccccttagacgcggttttagtagcggagccagtcagttcctgagacatgcccttagcgaggtaggttgccattttcgcagcgtctccaccccaggtagacacctgatcaagtttgaccccgtgctcacgcagtggcgcgtccataccggccttaaccacaccagcagaccagcgggaaaacatggaatcctcaaacgccttgagttcatcgtcagacagtggacgatccaagaacaacagcatgttgcggtgcaagtgccaaccgttcgcccaagagtctgtgacctcatagtcactataggtgtgctccaccccgtaccgtgcacgttctttcttccactgagatgttttcaccatcgaagagtacgcagtcttaatacccgcttcaacctgcgcaaatgactgtgagcggttgtgtcgaacagtgcccacaaacatcatgagcgcgccacccgccgccaagtgattcttagtagcaatagccagctcaatgcggcgttcgcccatgacttccaattcagccagaggtgacccccagcgagagtgagagttttgcagaccctcaaactgcgaagcaccgttagacgaccaggacaccgcaacagcttcgtccctgcgccacctatggcaccccgccagagccttactattggtgatcttgtacatgacgttttgcctacgccacgccctagcgcgagtgaccttagaaccctcattgacctgcggttccttagaggtgttcacttctatttcagtgttacctagacccgatgttgtgcggggttgcgcagtgcgagtttgtgcgggtgttgtgcccgttgtcttagctagtgctatggttgtcaattgaaaccccttcgggttatgtggcccccgtgcatatgagttggtagctcgcacgggggtttgtcttgtctaggaactattaatttttagtggtgtttggtggccgcctagcttggctatgcgtgccagcttacccgtactcaatgttaaagatttgcatcgacatgggagggttacgtgtccgatacctagggggggtatccgcgactaggtgccccggtgctcactgtctgtaccggcggggcaagccccacaccccgcatggacagggtggctccgccccctgcacccccagcaatctgcatgtacatgttttacacattagcacgacatgactgcatgtgcatgcactgcatgcagactaggtaaatatgagtatgtacgactagtaacaggagcactgcacataatgaatgagttgcaggacaatgtttgctacgcatgcgcatgacatatcgcaggaaagctactagagtcttaaagcatggcaaccaaggcacagctagaacagcaactacaagaagctcaacaggcactacaggcgcagcaagcgcaggcacaagccaccatcgaagcactagaagcgcaggcaaaggctaagcccgtcgtggtcaccgcacgcgttcctttggcactacgtgaggacatgaagcgcgcaggcatgcagaacggtgaaaacctccaagagttcatgatcgccgcgtttaccgagcggctagaaaagctcaccaccaccgacaacgaggaaaacaatgtctaacccactagttctctttgcccaccgtgacccggtaaatgacgtgacgttcgagtgcattgagcacgccacctacgacacactttcacacgctaaagaccagatcaccgcccaaatgcaagccctagacgaagaagccgccctactgccctaatgggtgtttcatgggtgtttccctagtgtttcatggtgttttcacctaagctagggaattgcgcgagaagtctcgcaaaaatcagcaacccccggaaccacacagttcacgggggttcttctatgccagaaatcagaaaggggaaccagtgaacgaccccgaatattggatcacagcgcagcaggtcgccgcccgcgtagctctcaccccggccaccattaaaaagtgggcaaacgagggaaaaatcaccgcatacaagatcggcaagtccgtccgattcaaagcatcagacgtagacaagctaggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcataacaccccttgtattactgtttatgtaagcagacagttttattgttcatgatgatatatttttatcttgtgcaatgtaacatcagagattttgagacacaacgtggctttccccccccccccaaaaggatctaggtgaagatcctttttgataatctcatgaccaaaatcccttaacgtgagttttcgttccactgagcgtcagaccccgtagaaaagatcaaaggatcttc

SEQ ID NO: 48 - DNA, 48 bp, Artificial Sequence
  Synthetic (MD-625)
tagggcgaattgggtaccatgattttgcatctgctgcgaaatctttgt

SEQ ID NO: 49 - DNA, 55 bp, Artificial Sequence
  Synthetic (MD-626)
gataccgtcgacctcgaggttgtgtctcctctaaagattgtaggaaatgcaatgt

SEQ ID NO: 50 - DNA, 48 bp, Artificial Sequence
  Synthetic (ncgl0049_up F)
catgattacgccaagcttcaatgcctttgatttgaactaagcacagga

SEQ ID NO: 51 - DNA, 43 bp, Artificial Sequence
  Synthetic (ncgl0049_up R)
tgcaaaatcattagagctcaaaaccgcggcgtaaaatattaga

SEQ ID NO: 52 - DNA, 31 bp, Artificial Sequence
  Synthetic (ncgl0049_down F)
tacggtgctagcatctgcccctttacaaatc

SEQ ID NO: 53 - DNA, 48 bp, Artificial Sequence
  Synthetic (ncgl0049_down R)
acgacggccagtgaattcggatcagccctaaatacaactatgagacag

SEQ ID NO: 54 - DNA, 42 bp, Artificial Sequence
  Synthetic (G3G F)
tgagctctaatgattttgcatctgctgcgaaatctttgtttc

SEQ ID NO: 55 - DNA, 32 bp, Artificial Sequence
  Synthetic (G3G R)
cagatgctagcaccgtatctgtggggggatgg

SEQ ID NO: 56 - DNA, 24 bp, Artificial Sequence
  Synthetic (cat1-seq1F)
agcccttactaagaaggttaatgc

SEQ ID NO: 57 - DNA, 22 bp, Artificial Sequence
  Synthetic (cat1-seq2R)
cgtacttgattgaaccatcgtt

SEQ ID NO: 58 - DNA, 22 bp, Artificial Sequence
  Synthetic (sucD-seq1F)
caatgttcaggtgatagtggac

SEQ ID NO: 59 - DNA, 20 bp, Artificial Sequence
  Synthetic (sucD-seq2R)
cacaacatctttcgcgatgg

SEQ ID NO: 60 - DNA, 21 bp, Artificial Sequence
  Synthetic (4hbD-seq1F)
ctgaccacgccataatcatcc

SEQ ID NO: 61 - DNA, 20 bp, Artificial Sequence
  Synthetic (4hbD-seq2R)
cgataccggcatagttgctg

SEQ ID NO: 62 - DNA, 37 bp, Artificial Sequence
  Synthetic (cat1 up)
ttgatatcgaattccatgtctaaaggaatcaagaata

SEQ ID NO: 63 - DNA, 35 bp, Artificial Sequence
  Synthetic (cat1 down)
tggcggccgctctagttacttcatcgagccagttt

SEQ ID NO: 64 - DNA, 35 bp, Artificial Sequence; identical to SEQ ID NO: 63
  Synthetic (sucD-up-IF)

SEQ ID NO: 65 - DNA, 35 bp, Artificial Sequence
  Synthetic (sucD-down-IF)
tggcggccgctctagttagagttcccagatttcct

SEQ ID NO: 66 - DNA, 35 bp, Artificial Sequence
  Synthetic (4hbD-up-IF)
ttgatatcgaattccatgcagcttttcaagctcaa

SEQ ID NO: 67 - DNA, 34 bp, Artificial Sequence
  Synthetic (4hbD-down-IF)
tggcggccgctctagctagtagagacgtcggtag

SEQ ID NO: 68 - DNA, 9334 bp, Artificial Sequence
  Synthetic (pC2E2)
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgcccctcttcgctattacgccagctggcgaaagggggatgtgctgcaaggcgattaagttgggtaacgccagggttttcccagtcacgacgttgtaaaacgacggccagtgaattgtaatacgactcactatagggcgaattggtgcgttaataaaggtggagaataagttgtttccaagatcaattcaaggaaagttgcattttcgcaggtcagtgttaccccctaagactacccctttccattgcatacaaaggaaatacatatagacttttgggcattagattacctcgataaaagtttagggaatctaaactcgagaggagacacaacatgaaggatgtactggcggaatacgcctcccgcattgtttcggcggaggaggccgttaagcacatcaaaaacggtgaacgggtagctttgtcacacgctgccggcgtgcctcagagttgcgttgacgcactggtgcagcaggccgaccttttccagaatgtggaaatctatcacatgctgtgcctcggtgagggtaagtatatggcgcctgagatggcccctcacttccgccacatcaccaactttgtcggtggtaactcccgtaaggcggtcgaagaaaaccgggccgatttcattccggtattcttttacgaggtgccaagcatgattcgcaaagacatcctccacattgatgtcgccatcgttcagctttcaatgcctgacgaaaatggttactgttcctttggagtatcttgcgattactccaagccggcagcagagagcgctcacctggttatcggagaaatcaaccgtcaaatgccatacgtacacggcgacaacttgattcatatctccaagttggattacatcgtgatggcagactaccccatctactctcttgcaaagcccaagatcggggaagtcgaggaagctatcgggaggaattgtgccgagcttattgaagatggtgccactctccagctgggaatcggcgcgattcctgatgcggccctgttatttctcaaggacaaaaaggatctgggcatccataccgaaatgttctccgatggtgttgtcgaattggttcgctccggcgttatcacaggcaagaaaaagactcttcaccccggaaagatggtcgcaaccttcctgatgggaagcgaggacgtgtatcatttcatcgacaaaaaccccgatgtagaactgtatccagtagattacgtgaatgacccgcgtgtgatcgcccaaaacgacaatatggtctcgattaacagctgcatcgaaatcgaccttatgggacaggtcgtgtccgagtgcatcggctcaaagcaattcagcggcaccggcggccaagttgactacgtgcgtggcgcagcatggtctaaaaacggcaagtccatcatggcaatcccgtccactgcaaaaaacggtacggcatctcgaattgtacctatcatcgcggagggcgctgctgtcaccaccctgcgcaacgaggtcgattacgttgtaaccgagtacggtatcgctcagctcaagggcaagagcctgcgccagcgcgcagaggctttgatcgcgatagcccaccccgacttccgtgaggaactaacgaaacatctccgcaagcgattcggagtctagatttcctacaatctttagaggagacacaacatgaaagtaaccaatcagaaagagttgaagcagaagttgaacgagctgcgagaggctcagaagaagttcgcaacctacacccaggaacaggtggacaagatctttaagcagtgtgccattgcagccgcgaaagaacgtattaatctcgcgaaacttgcggtcgaggaaaccggtattgggctggtagaagacaagatcatcaagaaccacttcgccgctgaatacatctacaacaagtacaaaaacgaaaagacatgtggtatcatcgaccacgacgacagcttgggcatcaccaaggtagcggagccaatcggtatcgtcgcagctatcgtgcccactactaaccctacctccactgctattttcaagtcactcatctccctgaaaacccgcaatgctatcttcttctcacctcacccacgcgctaagaaatcaactatcgcagcagctaaacttatcctggatgcagccgtgaaagccggggctccgaaaaacatcatcggttggatcgacgaaccttccattgaactctctcaagacctcatgtccgaggcagacattatcctggcaaccggaggcccatccatggttaaagcagcttacagctcaggcaagccggctatcggcgttggagctggtaacactccagcaatcatcgacgagtcggccgatatcgacatggcagtgtcctctattatcctgtccaaaacttatgacaacggcgttatttgcgcgtccgagcagtctattctcgtcatgaactctatttacgagaaggtaaaggaggagtttgtgaagcgggggtcgtacattctgaaccagaacgagatcgctaagatcaaagagactatgtttaaaaacggagccatcaacgcagatatcgtagggaagtccgcgtacatcattgctaagatggctggaatcgaagtccctcaaaccacgaaaattctgatcggcgaggtgcaatcggtcgaaaagtccgagctgttctcgcatgaaaagttgtccccggtcctcgcgatgtataaagttaaggattttgatgaagcactcaagaaagctcagcgcctgatcgaattgggtggctcgggtcacacctcttccctctacattgactcccagaacaataaagataaggtgaaagagttcggcctggctatgaagacgtctcgtaccttcatcaatatgccctcttcacagggcgccagcggtgacctttacaatttcgctatcgctcctagctttaccctcggctgcggcacctggggcggtaattctgtgtcccaaaacgtcgaaccaaagcatctgctcaacattaaaagcgtcgccgaacgtcgcgagaacatgttgtggttcaaggtcccgcaaaaaatctacttcaagtatggttgcttgcgctttgcacttaaagagcttaaggacatgaataaaaagcgggcgttcatcgtcactgataaggatctgttcaaactgggctatgttaacaagattaccaaggtcctggatgagatcgacatcaagtattccatcttcaccgatattaagtccgatccgaccattgattccgtgaagaagggcgcgaaggagatgctcaactttgaacccgacacgattatttctattggcggaggcagcccaatggacgcagctaaggttatgcacctgctgtatgagtacccagaagcagagatcgagaaccttgcaatcaatttcatggatattcgcaaacgcatttgcaactttcctaagctcggtacaaaagctatctctgttgcgatccctaccaccgcaggaaccggcagcgaagcgacaccattcgccgttattaccaacgatgaaacaggtatgaagtacccacttacctcttatgaacttaccccgaacatggctatcattgatacggaattgatgctgaacatgccacgcaagttgaccgcagctacgggaatcgacgcattggttcatgcaatcgaggcatacgtttccgtcatggcaaccgattacaccgacgaactcgcgttgcgtgcgattaaaatgatcttcaagtaccttccacgcgcatacaagaatggcacaaacgatattgaagcccgagaaaagatggcacacgcttcgaacatcgctggtatggccttcgcgaatgcgtttctcggagtgtgtcactccatggcgcacaaactgggagccatgcatcacgtgccccacggtatcgcatgcgccgttcttattgaagaggtgatcaagtataatgccaccgattgccccactaagcagacggccttccctcagtacaaatcgcccaatgccaagcgtaaatacgcggaaattgccgagtacttgaaccttaaggggaccagcgacacggaaaaggtgaccgcactgattgaagccatctccaagctcaagatcgacctgagcatcccacaaaacatctcagcagccggcattaacaagaaggacttctacaacactctcgacaagatgtcagaactcgccttcgatgatcagtgcactaccgcaaacccacgttatccgctcatctctgaactgaaggatatctacatcaagtcgttttagaattcatttagcggatgattctcgttcaacttcggccgaagccacttcgtctgtcataatgacagggatggtttcggccgtttttgcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccatccccccacagatacggtagctccagcttttgttccctttagtgagggttaatttcgagcttggcgtaatcatggtcatagctgtttcctgtgtgaaattgttatccgctcacaattccacacaacatacgagccggaagcataaagtgtaaagcctgggaacaacaagacccatcatagtttgcccccgcgacattgaccataaattcatcgcacaaaatatcgaacggggtttatgccgcttttagtgggtgcgaagaatagtctgctcattacccgcgaacaccgccgcattcagatcacgcttagtagcgtccccatgagtaggcagaaccgcgtccaagtccacatcatccataacgatcatgcacggggtggaatccacacccagacttgccagcacctcattagcgacacgttgcgcagcggccacgtccttagccttatccacgcaatctaggacgtactgcctaaccgcgaaatcagactgaatcagtttccaatcatcgggcttcaccaaagcaacagcaacgcgggttgattcgacccgttccggtgcttccagaccggcgagcttgtacagttcttcttccatttcacgacgtacatcagcgtctatgtaatcaatgcccaaagcacgcttagccccacgtgaccaggacgaacgcaggtttttagaaccaacctcatactcacgccaccgagccaccaaaacagcgtccatatcctcgccggcgtcgctttgatcggccaacatatccaacatctgaaacggcgtgtacgaccccttagacgcggttttagtagcggagccagtcagttcctgagacatgcccttagcgaggtaggttgccattttcgcagcgtctccaccccaggtagacacctgatcaagtttgaccccgtgctcacgcagtggcgcgtccataccggccttaaccacaccagcagaccagcgggaaaacatggaatcctcaaacgccttgagttcatcgtcagacagtggacgatccaagaacaacagcatgttgcggtgcaagtgccaaccgttcgcccaagagtctgtgacctcatagtcactataggtgtgctccaccccgtaccgtgcacgttctttcttccactgagatgttttcaccatcgaagagtacgcagtcttaatacccgcttcaacctgcgcaaatgactgtgagcggttgtgtcgaacagtgcccacaaacatcatgagcgcgccacccgccgccaagtgattcttagtagcaatagccagctcaatgcggcgttcgcccatgacttccaattcagccagaggtgacccccagcgagagtgagagttttgcagaccctcaaactgcgaagcaccgttagacgaccaggacaccgcaacagcttcgtccctgcgccacctatggcaccccgccagagccttactattggtgatcttgtacatgacgttttgcctacgccacgccctagcgcgagtgaccttagaaccctcattgacctgcggttccttagaggtgttcacttctatttcagtgttacctagacccgatgttgtgcggggttgcgcagtgcgagtttgtgcgggtgttgtgcccgttgtcttagctagtgctatggttgtcaattgaaaccccttcgggttatgtggcccccgtgcatatgagttggtagctcgcacgggggtttgtcttgtctaggaactattaatttttagtggtgtttggtggccgcctagcttggctatgcgtgccagcttacccgtactcaatgttaaagatttgcatcgacatgggagggttacgtgtccgatacctagggggggtatccgcgactaggtgccccggtgctcactgtctgtaccggcggggcaagccccacaccccgcatggacagggtggctccgccccctgcacccccagcaatctgcatgtacatgttttacacattagcacgacatgactgcatgtgcatgcactgcatgcagactaggtaaatatgagtatgtacgactagtaacaggagcactgcacataatgaatgagttgcaggacaatgtttgctacgcatgcgcatgacatatcgcaggaaagctactagagtcttaaagcatggcaaccaaggcacagctagaacagcaactacaagaagctcaacaggcactacaggcgcagcaagcgcaggcacaagccaccatcgaagcactagaagcgcaggcaaaggctaagcccgtcgtggtcaccgcacgcgttcctttggcactacgtgaggacatgaagcgcgcaggcatgcagaacggtgaaaacctccaagagttcatgatcgccgcgtttaccgagcggctagaaaagctcaccaccaccgacaacgaggaaaacaatgtctaacccactagttctctttgcccaccgtgacccggtaaatgacgtgacgttcgagtgcattgagcacgccacctacgacacactttcacacgctaaagaccagatcaccgcccaaatgcaagccctagacgaagaagccgccctactgccctaatgggtgtttcatgggtgtttccctagtgtttcatggtgttttcacctaagctagggaattgcgcgagaagtctcgcaaaaatcagcaacccccggaaccacacagttcacgggggttcttctatgccagaaatcagaaaggggaaccagtgaacgaccccgaatattggatcacagcgcagcaggtcgccgcccgcgtagctctcaccccggccaccattaaaaagtgggcaaacgagggaaaaatcaccgcatacaagatcggcaagtccgtccgattcaaagcatcagacgtagacaagctaggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcataacaccccttgtattactgtttatgtaagcagacagttttattgttcatgatgatatatttttatcttgtgcaatgtaacatcagagattttgagacacaacgtggctttccccccccccccaaaaggatctaggtgaagatcctttttgataatctcatgaccaaaatcccttaacgtgagttttcgttccactgagcgtcagaccccgtagaaaagatcaaaggatcttc

SEQ ID NO: 69 - DNA, 50 bp, Artificial Sequence
  Synthetic (P1)
ctatagggcgaattggtgcgttaataaaggtggagaataagttgtttcca

SEQ ID NO: 70 - DNA, 45 bp, Artificial Sequence
  Synthetic (P2)
gtctcctctcgagtttagattccctaaacttttatcgaggtaatc

SEQ ID NO: 71 - DNA, 6773 bp, Artificial Sequence
  Synthetic (MD0294)
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgcccctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccatgattttgcatctgctgcgaaatctttgtttccccgctaaagttgaggacaggttgacacggagttgactcgacgaattatccaatgtgagtaggtttggtgcgtgagttggaaaaattcgccatactcgcccttgggttctgtcagctcaagaattcttgagtgaccgatgctctgattgacctaactgcttgacacattgcatttcctacaatctttagaggagacacaacatgaaggatgtactggcggaatacgcctcccgcattgtttcggcggaggaggccgttaagcacatcaaaaacggtgaacgggtagctttgtcacacgctgccggcgtgcctcagagttgcgttgacgcactggtgcagcaggccgaccttttccagaatgtggaaatctatcacatgctgtgcctcggtgagggtaagtatatggcgcctgagatggcccctcacttccgccacatcaccaactttgtcggtggtaactcccgtaaggcggtcgaagaaaaccgggccgatttcattccggtattcttttacgaggtgccaagcatgattcgcaaagacatcctccacattgatgtcgccatcgttcagctttcaatgcctgacgaaaatggttactgttcctttggagtatcttgcgattactccaagccggcagcagagagcgctcacctggttatcggagaaatcaaccgtcaaatgccatacgtacacggcgacaacttgattcatatctccaagttggattacatcgtgatggcagactaccccatctactctcttgcaaagcccaagatcggggaagtcgaggaagctatcgggaggaattgtgccgagcttattgaagatggtgccactctccagctgggaatcggcgcgattcctgatgcggccctgttatttctcaaggacaaaaaggatctgggcatccataccgaaatgttctccgatggtgttgtcgaattggttcgctccggcgttatcacaggcaagaaaaagactcttcaccccggaaagatggtcgcaaccttcctgatgggaagcgaggacgtgtatcatttcatcgacaaaaaccccgatgtagaactgtatccagtagattacgtgaatgacccgcgtgtgatcgcccaaaacgacaatatggtctcgattaacagctgcatcgaaatcgaccttatgggacaggtcgtgtccgagtgcatcggctcaaagcaattcagcggcaccggcggccaagttgactacgtgcgtggcgcagcatggtctaaaaacggcaagtccatcatggcaatcccgtccactgcaaaaaacggtacggcatctcgaattgtacctatcatcgcggagggcgctgctgtcaccaccctgcgcaacgaggtcgattacgttgtaaccgagtacggtatcgctcagctcaagggcaagagcctgcgccagcgcgcagaggctttgatcgcgatagcccaccccgacttccgtgaggaactaacgaaacatctccgcaagcgattcggatagctcatttagcggatgattctcgttcaacttcggccgaagccacttcgtctgtcataatgacagggatggtttcggccgtttttgcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccatccccccacagatacggtagctccagcttttgttccctttagtgagggttaatttcgagcttggcgtaatcatggtcatagctgtttcctgtgtgaaattgttatccgctcacaattccacacaacatacgagccggaagcataaagtgtaaagcctgggaacaacaagacccatcatagtttgcccccgcgacattgaccataaattcatcgcacaaaatatcgaacggggtttatgccgcttttagtgggtgcgaagaatagtctgctcattacccgcgaacaccgccgcattcagatcacgcttagtagcgtccccatgagtaggcagaaccgcgtccaagtccacatcatccataacgatcatgcacggggtggaatccacacccagacttgccagcacctcattagcgacacgttgcgcagcggccacgtccttagccttatccacgcaatctaggacgtactgcctaaccgcgaaatcagactgaatcagtttccaatcatcgggcttcaccaaagcaacagcaacgcgggttgattcgacccgttccggtgcttccagaccggcgagcttgtacagttcttcttccatttcacgacgtacatcagcgtctatgtaatcaatgcccaaagcacgcttagccccacgtgaccaggacgaacgcaggtttttagaaccaacctcatactcacgccaccgagccaccaaaacagcgtccatatcctcgccggcgtcgctttgatcggccaacatatccaacatctgaaacggcgtgtacgaccccttagacgcggttttagtagcggagccagtcagttcctgagacatgcccttagcgaggtaggttgccattttcgcagcgtctccaccccaggtagacacctgatcaagtttgaccccgtgctcacgcagtggcgcgtccataccggccttaaccacaccagcagaccagcgggaaaacatggaatcctcaaacgccttgagttcatcgtcagacagtggacgatccaagaacaacagcatgttgcggtgcaagtgccaaccgttcgcccaagagtctgtgacctcatagtcactataggtgtgctccaccccgtaccgtgcacgttctttcttccactgagatgttttcaccatcgaagagtacgcagtcttaatacccgcttcaacctgcgcaaatgactgtgagcggttgtgtcgaacagtgcccacaaacatcatgagcgcgccacccgccgccaagtgattcttagtagcaatagccagctcaatgcggcgttcgcccatgacttccaattcagccagaggtgacccccagcgagagtgagagttttgcagaccctcaaactgcgaagcaccgttagacgaccaggacaccgcaacagcttcgtccctgcgccacctatggcaccccgccagagccttactattggtgatcttgtacatgacgttttgcctacgccacgccctagcgcgagtgaccttagaaccctcattgacctgcggttccttagaggtgttcacttctatttcagtgttacctagacccgatgttgtgcggggttgcgcagtgcgagtttgtgcgggtgttgtgcccgttgtcttagctagtgctatggttgtcaattgaaaccccttcgggttatgtggcccccgtgcatatgagttggtagctcgcacgggggtttgtcttgtctaggaactattaatttttagtggtgtttggtggccgcctagcttggctatgcgtgccagcttacccgtactcaatgttaaagatttgcatcgacatgggagggttacgtgtccgatacctagggggggtatccgcgactaggtgccccggtgctcactgtctgtaccggcggggcaagccccacaccccgcatggacagggtggctccgccccctgcacccccagcaatctgcatgtacatgttttacacattagcacgacatgactgcatgtgcatgcactgcatgcagactaggtaaatatgagtatgtacgactagtaacaggagcactgcacataatgaatgagttgcaggacaatgtttgctacgcatgcgcatgacatatcgcaggaaagctactagagtcttaaagcatggcaaccaaggcacagctagaacagcaactacaagaagctcaacaggcactacaggcgcagcaagcgcaggcacaagccaccatcgaagcactagaagcgcaggcaaaggctaagcccgtcgtggtcaccgcacgcgttcctttggcactacgtgaggacatgaagcgcgcaggcatgcagaacggtgaaaacctccaagagttcatgatcgccgcgtttaccgagcggctagaaaagctcaccaccaccgacaacgaggaaaacaatgtctaacccactagttctctttgcccaccgtgacccggtaaatgacgtgacgttcgagtgcattgagcacgccacctacgacacactttcacacgctaaagaccagatcaccgcccaaatgcaagccctagacgaagaagccgccctactgccctaatgggtgtttcatgggtgtttccctagtgtttcatggtgttttcacctaagctagggaattgcgcgagaagtctcgcaaaaatcagcaacccccggaaccacacagttcacgggggttcttctatgccagaaatcagaaaggggaaccagtgaacgaccccgaatattggatcacagcgcagcaggtcgccgcccgcgtagctctcaccccggccaccattaaaaagtgggcaaacgagggaaaaatcaccgcatacaagatcggcaagtccgtccgattcaaagcatcagacgtagacaagctaggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcataacaccccttgtattactgtttatgtaagcagacagttttattgttcatgatgatatatttttatcttgtgcaatgtaacatcagagattttgagacacaacgtggctttccccccccccccaaaaggatctaggtgaagatcctttttgataatctcatgaccaaaatcccttaacgtgagttttcgttccactgagcgtcagaccccgtagaaaagatcaaaggatcttc

SEQ ID NO: 72 - DNA, 37 bp, Artificial Sequence
  Synthetic (P3)
ctaaactcgagaggagacacaacatgaaggatgtact

SEQ ID NO: 73 - DNA, 35 bp, Artificial Sequence
  Synthetic (P4)
aggaaatctagactccgaatcgcttgcggagatgt

SEQ ID NO: 74 - DNA, 8054 bp, Artificial Sequence
  Synthetic (MD0295)
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgcccctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccatgattttgcatctgctgcgaaatctttgtttccccgctaaagttgaggacaggttgacacggagttgactcgacgaattatccaatgtgagtaggtttggtgcgtgagttggaaaaattcgccatactcgcccttgggttctgtcagctcaagaattcttgagtgaccgatgctctgattgacctaactgcttgacacattgcatttcctacaatctttagaggagacacaacatgaaagtaaccaatcagaaagagttgaagcagaagttgaacgagctgcgagaggctcagaagaagttcgcaacctacacccaggaacaggtggacaagatctttaagcagtgtgccattgcagccgcgaaagaacgtattaatctcgcgaaacttgcggtcgaggaaaccggtattgggctggtagaagacaagatcatcaagaaccacttcgccgctgaatacatctacaacaagtacaaaaacgaaaagacatgtggtatcatcgaccacgacgacagcttgggcatcaccaaggtagcggagccaatcggtatcgtcgcagctatcgtgcccactactaaccctacctccactgctattttcaagtcactcatctccctgaaaacccgcaatgctatcttcttctcacctcacccacgcgctaagaaatcaactatcgcagcagctaaacttatcctggatgcagccgtgaaagccggggctccgaaaaacatcatcggttggatcgacgaaccttccattgaactctctcaagacctcatgtccgaggcagacattatcctggcaaccggaggcccatccatggttaaagcagcttacagctcaggcaagccggctatcggcgttggagctggtaacactccagcaatcatcgacgagtcggccgatatcgacatggcagtgtcctctattatcctgtccaaaacttatgacaacggcgttatttgcgcgtccgagcagtctattctcgtcatgaactctatttacgagaaggtaaaggaggagtttgtgaagcgggggtcgtacattctgaaccagaacgagatcgctaagatcaaagagactatgtttaaaaacggagccatcaacgcagatatcgtagggaagtccgcgtacatcattgctaagatggctggaatcgaagtccctcaaaccacgaaaattctgatcggcgaggtgcaatcggtcgaaaagtccgagctgttctcgcatgaaaagttgtccccggtcctcgcgatgtataaagttaaggattttgatgaagcactcaagaaagctcagcgcctgatcgaattgggtggctcgggtcacacctcttccctctacattgactcccagaacaataaagataaggtgaaagagttcggcctggctatgaagacgtctcgtaccttcatcaatatgccctcttcacagggcgccagcggtgacctttacaatttcgctatcgctcctagctttaccctcggctgcggcacctggggcggtaattctgtgtcccaaaacgtcgaaccaaagcatctgctcaacattaaaagcgtcgccgaacgtcgcgagaacatgttgtggttcaaggtcccgcaaaaaatctacttcaagtatggttgcttgcgctttgcacttaaagagcttaaggacatgaataaaaagcgggcgttcatcgtcactgataaggatctgttcaaactgggctatgttaacaagattaccaaggtcctggatgagatcgacatcaagtattccatcttcaccgatattaagtccgatccgaccattgattccgtgaagaagggcgcgaaggagatgctcaactttgaacccgacacgattatttctattggcggaggcagcccaatggacgcagctaaggttatgcacctgctgtatgagtacccagaagcagagatcgagaaccttgcaatcaatttcatggatattcgcaaacgcatttgcaactttcctaagctcggtacaaaagctatctctgttgcgatccctaccaccgcaggaaccggcagcgaagcgacaccattcgccgttattaccaacgatgaaacaggtatgaagtacccacttacctcttatgaacttaccccgaacatggctatcattgatacggaattgatgctgaacatgccacgcaagttgaccgcagctacgggaatcgacgcattggttcatgcaatcgaggcatacgtttccgtcatggcaaccgattacaccgacgaactcgcgttgcgtgcgattaaaatgatcttcaagtaccttccacgcgcatacaagaatggcacaaacgatattgaagcccgagaaaagatggcacacgcttcgaacatcgctggtatggccttcgcgaatgcgtttctcggagtgtgtcactccatggcgcacaaactgggagccatgcatcacgtgccccacggtatcgcatgcgccgttcttattgaagaggtgatcaagtataatgccaccgattgccccactaagcagacggccttccctcagtacaaatcgcccaatgccaagcgtaaatacgcggaaattgccgagtacttgaaccttaaggggaccagcgacacggaaaaggtgaccgcactgattgaagccatctccaagctcaagatcgacctgagcatcccacaaaacatctcagcagccggcattaacaagaaggacttctacaacactctcgacaagatgtcagaactcgccttcgatgatcagtgcactaccgcaaacccacgttatccgctcatctctgaactgaaggatatctacatcaagtcgttttagctcatttagcggatgattctcgttcaacttcggccgaagccacttcgtctgtcataatgacagggatggtttcggccgtttttgcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccatccccccacagatacggtagctccagcttttgttccctttagtgagggttaatttcgagcttggcgtaatcatggtcatagctgtttcctgtgtgaaattgttatccgctcacaattccacacaacatacgagccggaagcataaagtgtaaagcctgggaacaacaagacccatcatagtttgcccccgcgacattgaccataaattcatcgcacaaaatatcgaacggggtttatgccgcttttagtgggtgcgaagaatagtctgctcattacccgcgaacaccgccgcattcagatcacgcttagtagcgtccccatgagtaggcagaaccgcgtccaagtccacatcatccataacgatcatgcacggggtggaatccacacccagacttgccagcacctcattagcgacacgttgcgcagcggccacgtccttagccttatccacgcaatctaggacgtactgcctaaccgcgaaatcagactgaatcagtttccaatcatcgggcttcaccaaagcaacagcaacgcgggttgattcgacccgttccggtgcttccagaccggcgagcttgtacagttcttcttccatttcacgacgtacatcagcgtctatgtaatcaatgcccaaagcacgcttagccccacgtgaccaggacgaacgcaggtttttagaaccaacctcatactcacgccaccgagccaccaaaacagcgtccatatcctcgccggcgtcgctttgatcggccaacatatccaacatctgaaacggcgtgtacgaccccttagacgcggttttagtagcggagccagtcagttcctgagacatgcccttagcgaggtaggttgccattttcgcagcgtctccaccccaggtagacacctgatcaagtttgaccccgtgctcacgcagtggcgcgtccataccggccttaaccacaccagcagaccagcgggaaaacatggaatcctcaaacgccttgagttcatcgtcagacagtggacgatccaagaacaacagcatgttgcggtgcaagtgccaaccgttcgcccaagagtctgtgacctcatagtcactataggtgtgctccaccccgtaccgtgcacgttctttcttccactgagatgttttcaccatcgaagagtacgcagtcttaatacccgcttcaacctgcgcaaatgactgtgagcggttgtgtcgaacagtgcccacaaacatcatgagcgcgccacccgccgccaagtgattcttagtagcaatagccagctcaatgcggcgttcgcccatgacttccaattcagccagaggtgacccccagcgagagtgagagttttgcagaccctcaaactgcgaagcaccgttagacgaccaggacaccgcaacagcttcgtccctgcgccacctatggcaccccgccagagccttactattggtgatcttgtacatgacgttttgcctacgccacgccctagcgcgagtgaccttagaaccctcattgacctgcggttccttagaggtgttcacttctatttcagtgttacctagacccgatgttgtgcggggttgcgcagtgcgagtttgtgcgggtgttgtgcccgttgtcttagctagtgctatggttgtcaattgaaaccccttcgggttatgtggcccccgtgcatatgagttggtagctcgcacgggggtttgtcttgtctaggaactattaatttttagtggtgtttggtggccgcctagcttggctatgcgtgccagcttacccgtactcaatgttaaagatttgcatcgacatgggagggttacgtgtccgatacctagggggggtatccgcgactaggtgccccggtgctcactgtctgtaccggcggggcaagccccacaccccgcatggacagggtggctccgccccctgcacccccagcaatctgcatgtacatgttttacacattagcacgacatgactgcatgtgcatgcactgcatgcagactaggtaaatatgagtatgtacgactagtaacaggagcactgcacataatgaatgagttgcaggacaatgtttgctacgcatgcgcatgacatatcgcaggaaagctactagagtcttaaagcatggcaaccaaggcacagctagaacagcaactacaagaagctcaacaggcactacaggcgcagcaagcgcaggcacaagccaccatcgaagcactagaagcgcaggcaaaggctaagcccgtcgtggtcaccgcacgcgttcctttggcactacgtgaggacatgaagcgcgcaggcatgcagaacggtgaaaacctccaagagttcatgatcgccgcgtttaccgagcggctagaaaagctcaccaccaccgacaacgaggaaaacaatgtctaacccactagttctctttgcccaccgtgacccggtaaatgacgtgacgttcgagtgcattgagcacgccacctacgacacactttcacacgctaaagaccagatcaccgcccaaatgcaagccctagacgaagaagccgccctactgccctaatgggtgtttcatgggtgtttccctagtgtttcatggtgttttcacctaagctagggaattgcgcgagaagtctcgcaaaaatcagcaacccccggaaccacacagttcacgggggttcttctatgccagaaatcagaaaggggaaccagtgaacgaccccgaatattggatcacagcgcagcaggtcgccgcccgcgtagctctcaccccggccaccattaaaaagtgggcaaacgagggaaaaatcaccgcatacaagatcggcaagtccgtccgattcaaagcatcagacgtagacaagctaggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcataacaccccttgtattactgtttatgtaagcagacagttttattgttcatgatgatatatttttatcttgtgcaatgtaacatcagagattttgagacacaacgtggctttccccccccccccaaaaggatctaggtgaagatcctttttgataatctcatgaccaaaatcccttaacgtgagttttcgttccactgagcgtcagaccccgtagaaaagatcaaaggatcttc

SEQ ID NO: 75 - DNA, 48 bp, Artificial Sequence
  Synthetic (P5)
ttcggagtctagatttcctacaatctttagaggagacacaacatgaaa

SEQ ID NO: 76 - DNA, 47 bp, Artificial Sequence
  Synthetic (P6)
ccgctaaatgaattctaaaacgacttgatgtagatatccttcagttc

SEQ ID NO: 77 - DNA, 42 bp, Artificial Sequence
  Synthetic (P7)
cgttttagaattcatttagcggatgattctcgttcaacttcg

SEQ ID NO: 78 - DNA, 43 bp, Artificial Sequence
  Synthetic (P8)
gaacaaaagctggagctaccgtatctgtggggggatggcttgt

SEQ ID NO: 79 - DNA, 20 bp, Artificial Sequence
  Synthetic (P9 primer)
accgtatctgtggggggatg

SEQ ID NO: 80 - DNA, 8031 bp, Artificial Sequence
  Synthetic (pG2G vector)
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgcccctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccatgattttgcatctgctgcgaaatctttgtttccccgctaaagttgaggacaggttgacacggagttgactcgacgaattatccaatgtgagtaggtttggtgcgtgagttggaaaaattcgccatactcgcccttgggttctgtcagctcaagaattcttgagtgaccgatgctctgattgacctaactgcttgacacattgcatttcctacaatctttagaggagacacaacctcgaggtcgacggtatcgataagcttgatatcgaattccatggagattaaagagatggtcagtcttgcgcgcaaagctcagaaggagtatcaggccacccataaccaagaagctgtggacaacatctgccgagctgcagcgaaggttatttacgaaaatgcagcaattctggcccgcgaggcagtggacgaaaccggcatgggtgtttacgagcacaaggtggccaagaatcaaggcaagtccaaaggtgtttggtacaacctgcataacaagaagtcgattggcatcctcaatatcgatgagcgtaccggcatgatcgagatcgcaaaacctatcggggttgtaggcgccgttacgccaaccaccaaccctatcgttactccgatgagcaacatcatctttgctcttaagacctgcaacgccatcattatcgccccacacccgcgctccaaaaagtgctctgcccacgcagttcggctgatcaaagaggctatcgctccgttcaacgtgcccgaaggtatggttcagatcatcgaggagcctagcatcgagaagacgcaggaattgatgggcgccgtagacgtggtcgttgctaccgggggcatgggcatggtcaagtctgcctactcctcagggaagccttctttcggtgtcggagccggcaatgttcaggtgatagtggacagcaacatcgatttcgaagcggctgcagaaaagatcatcaccggacgtgccttcgacaacggtatcatctgctcaggcgaacagtccatcatctacaacgaggctgacaaggaagcagttttcacagcattccgcaaccacggtgcgtacttttgcgacgaggccgagggagatcgggctcgtgcagcgatcttcgaaaatggagccatcgcgaaagatgttgtgggccagtccgttgcctttattgccaagaaggcgaacattaatatccccgagggtactcgtattctcgtggtcgaagctcgcggagtaggcgccgaagatgtcatctgtaaagaaaagatgtgtccagtcatgtgcgccctctcctacaagcacttcgaagagggggtagagatcgcaaggacgaacctcgcaaacgaaggcaatggccatacctgtgctatccactccaacaaccaagcacacatcatcttggcaggctcggagctgaccgtgtctcgcatcgtggtcaacgcgccaagtgctaccacagcaggcggtcacatccagaacggtcttgccgtcaccaatactctaggctgcggctcttggggtaacaactcgatctccgaaaacttcacttataaacacctgctcaacatttcacgcatcgccccgttgaactccagcattcatatcccagatgataaggaaatctgggaactctaattagaggagacacaacatgcagcttttcaagctcaagagcgtcacacatcactttgatacttttgcagagtttgccaaggaattctgtctcggtgaacgcgacttggtaattaccaacgagttcatctacgaaccgtatatgaaggcatgccagctgccttgtcattttgtgatgcaggagaaatacggccaaggcgagccttctgacgagatgatgaacaacatcctagcagatatccgtaatatccagttcgaccgcgtgatcgggatcggaggtggtacggttattgacatctcaaaactctttgttctgaagggattaaatgatgttctcgacgcgttcgatcgcaagattccccttatcaaagagaaagaactgatcattgtgcccaccacctgcggaaccggctcggaggtgacgaacatttccatcgccgagatcaagtcccggcacaccaagatgggtttggctgacgatgcaattgttgctgaccacgccataatcatccctgaacttctgaagagcttgcccttccacttctatgcatgctccgcaatcgatgctcttattcatgccatcgagtcatacgtttctccaaaagcgtctccatactcccgtctgttcagtgaggcggcgtgggacattatcctggaagttttcaagaaaatcgccgaacacggcccagagtaccgcttcgagaagctgggggaaatgatcatggccagcaactatgccggtatcgctttcggcaacgcaggcgttggcgccgtccacgctctatcctacccgttgggcggcaactatcacgtgccgcatggagaagcaaactatcagttcttcaccgaggtctttaaagtataccaaaagaagaatccgttcggctatattgtcgaactcaactggaagctctccaagattctgaactgccagccagagtacgtgtacccgaagctggatgaactgctcggttgccttcttaccaagaaacctttgcacgaatacggcatgaaggacgaagaggttcgtggcttcgcggaatcggtcctgaagacccagcaacgcttgctcgccaacaactacgtcgaacttactgtcgatgagatcgaaggtatctaccgacgtctctactaactagagcggccgccaccgcggtggagctcatttagcggatgattctcgttcaacttcggccgaagccacttcgtctgtcataatgacagggatggtttcggccgtttttgcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccatccccccacagatacggtagctccagcttttgttccctttagtgagggttaatttcgagcttggcgtaatcatggtcatagctgtttcctgtgtgaaattgttatccgctcacaattccacacaacatacgagccggaagcataaagtgtaaagcctgggaacaacaagacccatcatagtttgcccccgcgacattgaccataaattcatcgcacaaaatatcgaacggggtttatgccgcttttagtgggtgcgaagaatagtctgctcattacccgcgaacaccgccgcattcagatcacgcttagtagcgtccccatgagtaggcagaaccgcgtccaagtccacatcatccataacgatcatgcacggggtggaatccacacccagacttgccagcacctcattagcgacacgttgcgcagcggccacgtccttagccttatccacgcaatctaggacgtactgcctaaccgcgaaatcagactgaatcagtttccaatcatcgggcttcaccaaagcaacagcaacgcgggttgattcgacccgttccggtgcttccagaccggcgagcttgtacagttcttcttccatttcacgacgtacatcagcgtctatgtaatcaatgcccaaagcacgcttagccccacgtgaccaggacgaacgcaggtttttagaaccaacctcatactcacgccaccgagccaccaaaacagcgtccatatcctcgccggcgtcgctttgatcggccaacatatccaacatctgaaacggcgtgtacgaccccttagacgcggttttagtagcggagccagtcagttcctgagacatgcccttagcgaggtaggttgccattttcgcagcgtctccaccccaggtagacacctgatcaagtttgaccccgtgctcacgcagtggcgcgtccataccggccttaaccacaccagcagaccagcgggaaaacatggaatcctcaaacgccttgagttcatcgtcagacagtggacgatccaagaacaacagcatgttgcggtgcaagtgccaaccgttcgcccaagagtctgtgacctcatagtcactataggtgtgctccaccccgtaccgtgcacgttctttcttccactgagatgttttcaccatcgaagagtacgcagtcttaatacccgcttcaacctgcgcaaatgactgtgagcggttgtgtcgaacagtgcccacaaacatcatgagcgcgccacccgccgccaagtgattcttagtagcaatagccagctcaatgcggcgttcgcccatgacttccaattcagccagaggtgacccccagcgagagtgagagttttgcagaccctcaaactgcgaagcaccgttagacgaccaggacaccgcaacagcttcgtccctgcgccacctatggcaccccgccagagccttactattggtgatcttgtacatgacgttttgcctacgccacgccctagcgcgagtgaccttagaaccctcattgacctgcggttccttagaggtgttcacttctatttcagtgttacctagacccgatgttgtgcggggttgcgcagtgcgagtttgtgcgggtgttgtgcccgttgtcttagctagtgctatggttgtcaattgaaaccccttcgggttatgtggcccccgtgcatatgagttggtagctcgcacgggggtttgtcttgtctaggaactattaatttttagtggtgtttggtggccgcctagcttggctatgcgtgccagcttacccgtactcaatgttaaagatttgcatcgacatgggagggttacgtgtccgatacctagggggggtatccgcgactaggtgccccggtgctcactgtctgtaccggcggggcaagccccacaccccgcatggacagggtggctccgccccctgcacccccagcaatctgcatgtacatgttttacacattagcacgacatgactgcatgtgcatgcactgcatgcagactaggtaaatatgagtatgtacgactagtaacaggagcactgcacataatgaatgagttgcaggacaatgtttgctacgcatgcgcatgacatatcgcaggaaagctactagagtcttaaagcatggcaaccaaggcacagctagaacagcaactacaagaagctcaacaggcactacaggcgcagcaagcgcaggcacaagccaccatcgaagcactagaagcgcaggcaaaggctaagcccgtcgtggtcaccgcacgcgttcctttggcactacgtgaggacatgaagcgcgcaggcatgcagaacggtgaaaacctccaagagttcatgatcgccgcgtttaccgagcggctagaaaagctcaccaccaccgacaacgaggaaaacaatgtctaacccactagttctctttgcccaccgtgacccggtaaatgacgtgacgttcgagtgcattgagcacgccacctacgacacactttcacacgctaaagaccagatcaccgcccaaatgcaagccctagacgaagaagccgccctactgccctaatgggtgtttcatgggtgtttccctagtgtttcatggtgttttcacctaagctagggaattgcgcgagaagtctcgcaaaaatcagcaacccccggaaccacacagttcacgggggttcttctatgccagaaatcagaaaggggaaccagtgaacgaccccgaatattggatcacagcgcagcaggtcgccgcccgcgtagctctcaccccggccaccattaaaaagtgggcaaacgagggaaaaatcaccgcatacaagatcggcaagtccgtccgattcaaagcatcagacgtagacaagctaggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcataacaccccttgtattactgtttatgtaagcagacagttttattgttcatgatgatatatttttatcttgtgcaatgtaacatcagagattttgagacacaacgtggctttccccccccccccaaaaggatctaggtgaagatcctttttgataatctcatgaccaaaatcccttaacgtgagttttcgttccactgagcgtcagaccccgtagaaaagatcaaaggatcttc

SEQ ID NO: 81 - DNA, 42 bp, Artificial Sequence
  Synthetic (P10 primer)
catccccccacagatacggtatgattttgcatctgctgcgaa

SEQ ID NO: 82 - protein, 502 aa, Corynebacterium glutamicum
MSDRIASEKLRSKLMSADEAAQFVNHGDKVGFSGFTGAGYPKALPTAIANRAKEAHGAGNDYAIDLFTGASTAPDCDGVLAEADAIRWRMPYASDPIMRNKINSGSMGYSDIHLSHSGQQVEEGFFGQLNVAVIEITRITEEGYIIPSSSVGNNVEWLNAAEKVILEVNSWQSEDLEGMHDIWSVPALPNRIAVPINKPGDRIGKTYIEFDTDKVVAVVETNTADRNAPFKPVDDISKKIAGNFLDFLESEVAAGRLSYDGYIMQSGVGNVPNAVMAGLLESKFENIQAYTEVIQDGMVDLIDAGKMTVASATSFSLSPEYAEKMNNEAKRYRESIILRPQQISNHPEVIRRVGLIATNGLIEADIYGNVNSTNVSGSRVMNGIGGSGDFTRNGYISSFITPSEAKGGAISAIVPFASHIDHTEHDVMVVISEYGYADLRGLAPRERVAKMIGLAHPDYRPLLEEYYARATSGDNKYMQTPHDLATAFDFHINLAKNGSMKA

SEQ ID NO: 83 - DNA, 1509 bp, Corynebacterium glutamicum
atgtctgatcgcattgcttcagaaaagctgcgctccaagctcatgtccgccgacgaggcggcacagtttgttaaccacggtgacaaggttggtttctccggcttcaccggcgctggctacccaaaggcactgcctacggcaatcgctaaccgggctaaagaagcacacggtgcaggcaacgactacgcaatcgacctgttcactggcgcatcgaccgcccctgactgcgatggcgtacttgcagaagctgacgctatccgctggcgcatgccatacgcatctgatccaatcatgcgtaacaagatcaactccggctccatgggatactccgatatccacctgtcccactccggccagcaggttgaagagggcttcttcggccagctcaacgtagctgtcattgaaatcacccgcatcactgaagagggctacatcatcccttcttcctccgtgggtaacaacgttgagtggctcaacgctgcagagaaggtcatcctcgaggttaactcttggcagtctgaagacctcgaaggtatgcacgacatctggtctgttcctgccctgccaaaccgcattgccgtgccaatcaacaagccaggcgaccgcatcggtaagacctacatcgagttcgacaccgacaaggttgttgctgttgttgagaccaacaccgcagaccgcaacgcaccattcaagcctgtcgacgacatctctaagaagatcgctggcaacttcctcgacttcctggaaagcgaagttgctgcaggtcgcctgtcctacgacggctacatcatgcagtccggcgtgggcaacgtgccaaacgcggtgatggcaggcctgctggaatccaagtttgagaacatccaggcctacaccgaagttatccaggacggcatggtggacctcatcgacgccggcaagatgaccgttgcatccgcaacttccttctccctgtctcctgagtacgcagagaagatgaacaacgaggctaagcgttaccgcgagtccattatcctgcgcccacagcagatctctaaccacccagaggtcatccgccgcgttggcctgatcgccaccaacggtctcatcgaggctgacatttacggcaacgtcaactccaccaacgtttctggctcccgcgtcatgaacggcatcggcggctccggcgacttcacccgtaacggctacatctccagcttcatcaccccttcagaggcaaagggcggcgcaatctctgcgatcgttcctttcgcatcccacatcgaccacaccgagcacgatgtcatggttgttatctctgagtacggttacgcagaccttcgtggtctggctccacgtgagcgcgttgccaagatgatcggcctggctcaccctgattaccgcccactgctcgaggagtactacgctcgcgcaacctccggtgacaacaagtacatgcagacccctcatgatcttgcaaccgcgtttgatttccacatcaacctggctaagaacggctccatgaaggcataa

SEQ ID NO: 84 - protein, 125 aa, Corynebacterium glutamicum
MNGIGGSGDFTRNAFASTFISPSAAKVDAISAIVPFASHIDHTEHDAMVVITEYGYADLRGLSPKQRVPKMIAIAHPDYRPLLEAYFDRALNSADSYQHTLHDLRTAFDFHNRLNSQGTMKIEKA

SEQ ID NO: 85 - DNA, 378 bp, Corynebacterium glutamicum
atgaatggtatcggcggctcgggcgatttcacgcgtaacgcctttgcttccacatttatctctccctcggcagccaaagttgatgcgatttccgcgattgtgcctttcgcgtcccatatcgatcacacggaacatgatgcgatggttgtcattactgaatatggctacgcagacctgcgcgggctatcgccaaaacaacgagtccccaaaatgattgccatcgcccacccggactatcgaccactgctggaagcatactttgaccgggcgctgaacagtgctgattcctatcagcacaccctgcatgatctgcgcaccgccttcgatttccataatcgcttgaactcacaaggaaccatgaaaatcgaaaaagcatag

(19) United States
(12) Patent Application Publication
(10) Pub. No.: US 2015/0004661 A1
(43) Pub. Date: Jan. 1, 2015

(54) CORYNEBACTERIUM FOR PRODUCING C4 COMPOUNDS AND METHOD OF PRODUCING C4 CHEMICALS BY USING THE SAME

(71) Applicant: Samsung Electronics Co., Ltd., Suwon-si (KR)

(72) Inventors: Hong-soon RHEE, Hwaseong-si (KR); Young-min Lee, Suwon-si (KR); Jin-woo Kim, Seoul (KR); Eun-Young Park, Yongin-si (KR); Yeo-ju Song, Incheon (KR); Jin-hwan Park, Suwon-si (KR); Kwang-myung Cho, Seongnam-si (KR)

(21) Appl. No.: 14/319,116

(22) Filed: Jun. 30, 2014

(30) Foreign Application Priority Data
Jun. 28, 2013 (KR) 10-2013-0075940
Jul. 30, 2013 (KR) 10-2013-0090441

(51) Int. Cl. C12P 7/42; C12P 17/04; C12P 7/18; C12P 7/46; C12P 7/40

(52) U.S. Cl. CPC C12P 7/42; C12P 7/46; C12P 7/40; C12P 7/18; C12P 17/04
USPC 435/126; 435/252.32; 435/145; 435/136; 435/158; 435/146

(57) ABSTRACT

A microorganism expressing a vector encoding a CoA-dependent succinate semialdehyde dehydrogenase to efficiently produce a C4 compound, and methods for the use thereof.

### RELATED APPLICATION

This application claims the benefit of Korean Patent Application No. 10-2013-0075940 filed on Jun. 28, 2013, and Korean Patent Application No. 10-2013-0090441 filed on Jul. 30, 2013 in the Korean Intellectual Property Office, the entire disclosures of which are hereby incorporated by reference.

### INCORPORATION-BY-REFERENCE OF MATERIAL ELECTRONICALLY SUBMITTED

Incorporated by reference in its entirety herein is a computer-readable nucleotide/amino acid sequence listing submitted herewith and identified as follows: 145,968 bytes ASCII (Text) file named “718130_ST25.TXT,” created Jun. 30, 2014.

### BACKGROUND

1. Field

The present disclosure relates to microorganisms in which vectors encoding CoA-dependent succinate semialdehyde dehydrogenases are expressed and methods of producing metabolites such as C4 compounds by using the same.

2. Description of the Related Art

Bioplastic is a polymer synthesized by using biomass as a raw material, which is derived from a renewable plant instead of a conventional fossil fuel. Since the biomass is a raw material that requires carbon dioxide in the air during a biosynthesis by a photosynthesis process, bioplastic is a very useful material to reduce carbon emission. Bioplastic replaces fossil fuel, reduces carbon dioxide, and does not cause environmental problems, and thus, bioplastic may be widely used in the low carbon green growth industry.

The biodegradable polymer material is being suggested as an alternative material that may replace a synthetic polymer material. 4-hydroxybutyrate shows a wide range of properties from crystalline plastic to highly elastic rubber and thus, lots of research is being conducted into a microorganism degradable plastic. 4-hydroxybutyrate may be converted into 1,4-butanediol and 1,4-butanediol is used in the whole chemical industry such as in the synthesis of a polymer, a solvent, and an intermediate material used in fine chemistry. However, considering the increase in production cost according to the increase in the price of oil, there is a need for a biological production method that may supplement or substitute chemical processes.

To this end, a method of producing C4-compound such as 4-hydroxybutyrate by using a transformed microorganism and a highly concentrated chemical material converted therefrom is needed.

### SUMMARY

According to an embodiment, provided is a microorganism that produces a C4 compound, wherein the microorganism comprises a vector encoding a CoA-dependent succinate semialdehyde and expresses a CoA-dependent succinate semialdehyde from the vector.

According to another embodiment, provided is a method of producing a C4 compound by using the microorganism that expresses the CoA-dependent succinate semialdehyde dehydrogenase from the vector.

According to another embodiment, provided is a microorganism in which a gene coding for a CoA-independent succinic semialdehyde dehydrogenase is inactivated or attenuated.

According to another embodiment, provided is a method of producing a C4 compound using the microorganism in which a gene coding for a CoA-independent succinic semialdehyde dehydrogenase is inactivated or attenuated.

Additional aspects will be set forth in part in the description which follows and, in part, will be apparent from the description, or may be learned by practice of the presented embodiments.

### BRIEF DESCRIPTION OF THE DRAWINGS

These and/or other aspects will become apparent and more readily appreciated from the following description of the embodiments, taken in conjunction with the accompanying drawings in which:

FIG. 1 is a cleavage map of a pGSK+ vector;

FIG. 2 is a cleavage map of a pGT1 vector;

FIG. 3 is a cleavage map of a pG3G vector;

FIG. 4 is a cleavage map of a pGSX1 vector;

FIG. 5 is a cleavage map of a pG2G vector;

FIG. 6 shows productivity of 4-hydroxybutyrate (4HB) in B039 pG3G and B039 pG2G;

FIG. 7 shows productivity of 4HB of the strains disclosed in Table 2;

FIG. 8 shows productivity of 4HB and 1,4-BDO in D003, D003 pC2E2, D001, and D001 pC2E2;

FIG. 9 is a cleavage map of a pcat1 vector;

FIG. 10 is a cleavage map of a psucD vector;

FIG. 11 is a cleavage map of a p4hbD vector;

FIG. 12 shows productivity of 4HB in the strains disclosed in Table 4;

FIG. 13 shows productivity of 4HB in D003 and D003 pG2G;

FIG. 14 shows productivity of 4HB according to culturing conditions;

FIG. 15 shows an example of a biosynthetic pathway for producing 4HB from glucose;

FIG. 16 is a cleavage map of a pC2E2 vector.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description.

According to an embodiment, provided is a microorganism producing a C4 compound (4-carbon compound), in which the microorganism expresses a CoA-dependent succinate semialdehyde dehydrogenase from a gene in a vector.

The microorganism may be any one selected from the group consisting of Lumen bacteria, Corynebacterium genus, and Brevibacterium genus. The Corynebacterium genus microorganism may be a microorganism selected from the group consisting of Corynebacterium glutamicum, Corynebacterium thermoaminogenes, Brevibacterium flavum, and Brevibacterium lactofermentum.

The CoA-dependent succinate semialdehyde dehydrogenase may be an enzyme having catalytic activity for converting succinyl-CoA into succinic semialdehyde. The enzyme may be categorized as EC.1.2.1.b. For example, the enzyme may be SucD. SucD may have an amino acid sequence of SEQ ID NO: 1 derived from Porphyromonas gingivalis. A polynucleotide coding for SucD may code for an amino acid sequence of SEQ ID NO: 1. The polynucleotide coding for SucD may have a nucleotide sequence of SEQ ID NO: 2 derived from Porphyromonas gingivalis.

The term “vector” refers to a DNA construct including a DNA sequence that is operably linked to a suitable regulatory sequence that may express DNA in a suitable host. The vector is physically separated from chromosomal DNA (may also be referred to as ‘genomic DNA’ hereinafter) of a subject and in some cases, the vector may be replicated independently from the chromosome. The vector may include a plasmid vector, a bacteriophage vector, and a cosmid vector. To operate as a vector expressing a gene, the vector may include an origin of replication, a promoter, a multiple cloning site (MCS), and a selection marker. The origin of replication enables the vector to be replicated separately from a chromosome of a host cell, the promoter acts in transcription of a gene to be inserted, and the MCS provides various restriction sites in which exotic genes may be inserted. The selection marker acts to confirm the insertion of a vector into a host cell. The selection marker includes an antibiotic resistant gene that is generally used in the art. The selection marker may have a resistance gene for, for example, ampicillin, gentamicin, carbenicillin, chloramphenicol, streptomycin, kanamycin, geneticin, neomycin, or tetracycline.

When a prokaryotic cell is used as a host, the vector may include a strong promoter, for example, a lambda PL promoter, a trp promoter, a lac promoter, a T7 promoter, or a tac promoter. When a eukaryotic cell is used as a host, the vector may include a promoter derived from a genome of a mammalian cell, for example, a metalllothionein promoter, or a promoter derived from a mammalian virus, for example, an adenovirus late promoter, a vaccinia virus 7.5K promoter, an SV40 promoter, a cytomegalovirus promoter, and a tk promoter of HSV. The promoter may be a lambda PL promoter, a trp promoter, a lac promoter, a T7 promoter, or a tac promoter. The promoter is operably linked to a sequence coding for a gene.

Also, the promoter is operably linked to a sequence coding for a gene. The expression, “operably linked” refers to a functional linkage between a nucleic acid expression regulatory sequence, for example, a promoter, a signal sequence, an array at a binding location of a transcription factor, a terminator, or an enhancer, and another nucleotide sequence and thus, the regulatory sequence may regulate transcription and/or translation of the nucleotide sequence coding for the gene.

The vector may include a CoA-dependent succinate semialdehyde dehydrogenase gene that is operably linked to a regulatory sequence that is different from the CoA-dependent succinate semialdehde dehydrogenase gene in the chromosome.

The microorganism producing the C4 compound may be a microorganism having inactivated or reduced activity of a pathway for synthesizing lactate from pyruvate. The microorganism may have an inactivated or attenuated gene coding an L-lactate dehydrogenase (LDH). The LDH may have a catalytic activity for converting pyruvate into lactate. The LDH may be an enzyme categorized as EC.1.1.1.27. The LDH may have an amino acid sequence of SEQ ID NO: 3. The gene coding for LDH may be a gene coding for an amino acid sequence of SEQ ID NO: 3.

The term “inactivation” or “inactivated” may mean that a gene is not expressed or a gene is expressed but a product of the expressed gene is not active. The term “attenuation” or “attenuated” may mean that the expression of a gene is decreased to a level lower than an expression level of a wild type strain, a strain which is not genetically engineered, or a parent strain. Alternatively, “attenuation” may mean that a product of the expression of the gene has an attenuated activity. Inactivation and attenuation encompasses deletion of a gene in whole or in part. The inactivation or attenuation may be performed, for example, by homologous recombination, site directed mutagenesis etc. The inactivation or attenuation may include changing a expression regulatory sequence such as the sequence of promoter, transcription terminator, operator, and the like by a site specific mutation method, or homologous recombination method. The inactivation or attenuation may include change the coding sequence of a gene by a site specific mutation method, or homologous recombination method. The inactivation or attenuation may be caused by insertion, substitution, conversion, or addition of nucleotide(s). The inactivation or attenuation may be performed, for example, by transforming a vector including a part of the sequence of the genes into a cell, culturing the cell so that homologous recombination of the sequence may occur with a homologous gene of the cell, and then using a selection marker to select a cell in which homologous recombination occurred. Due to inactivation or attenuation of the gene, activity of the enzyme coded from the gene may be eliminated or decreased. The term “decreased” may refer to relative activity of a manipulated microorganism compared with an unmanipulated microorganism, i.e. a parent cell.

The microorganism producing a C4 compound may have an inactivated or attenuated gene for a CoA independent succinic semialdehyde dehydrogenase (SSADH). The SSADH may have activity for catalyzing a reaction that converts succinic semialdehyde into succinate. The SSADH may be an enzyme categorized as EC.1.2.1.16 or EC.1.2.1.39. The SSADH may be NAD+ or NADP+ dependent. The functional form of SSADH may include an amino acid sequence of SEQ ID NO: 4, SEQ ID NO: 5, or SEQ ID NO: 6. A gene coding for the SSADH may include a nucleotide sequence of SEQ ID NO: 7, SEQ ID NO: 8, or SEQ ID NO: 9. The microorganism may be a microorganism in which the gene coding for the SSADH is inactivated or attenuated.

Also, the microorganism producing a C4 compound in which a CoA-dependent succinate semialdehyde dehydrogenase is expressed from a gene in a vector may additionally include a polynucleotide coding for a 4-hydroxybutyrate dehydrogenase in a vector.

The 4-hydroxybutyrate dehydrogenase may be an enzyme having a catalytic activity for converting succinic semialdehyde into 4-hydroxybutyrate. The enzyme may be an enzyme categorized as EC.1.1.1.a. For example, the enzyme may be 4hbD. The 4hbD may have an amino acid sequence of SEQ ID NO: 10 derived from Porhyromonas gingivalis. A polynucleotide coding for 4hbD may code for an amino acid sequence of SEQ ID NO: 10. The polynucleotide coding for the 4hbD may have a nucleotide sequence of SEQ ID NO: 11.

Also, the microorganism may include a polynucleotide coding for a CoA-dependent succinate semialdehyde dehydrogenase and a polynucleotide coding for a 4-hydroxybutyrate dehydrogenase in a chromosome of the microorganism, apart from the CoA-dependent succinate semialdehyde dehydrogenase and the 4-hydroxybutyrate dehydrogenase expressed each gene present in a vector.

Also, the microorganism may additionally include a polynucleotide coding for a succinyl-CoA:CoA transferase. The polynucleotide coding for a succinyl-CoA:CoA transferase may exist in a chromosome or a vector.

The succinyl-CoA:CoA transferase may be an enzyme having an activity for catalyzing a reaction that converts succiinate into succinyl-CoA. The enzyme may be an enzyme categorized as EC.2.8.3.a. For example, the enzyme may be Cat1. The Cat1 may have an amino acid sequence of SEQ ID NO: 12 derived from Clostridium kluyveri. A polynucleotide coding for Cat1 may code for an amino acid sequence of SEQ ID NO: 12. The polynucleotide coding for Cat1 may have a nucleotide sequence of SEQ ID NO: 13.

The polynucleotide coding for the succinyl-CoA:CoA transferase may be located in a chromosome of the microorganism.

Accordingly, the microorganism may include all of the polynucleotide coding for the succinyl-CoA:CoA transferase, the polynucleotide coding for the CoA-dependent succinate semialdehyde dehydrogenase, and the polynucleotide coding for the 4-hydroxybutyrate dehydrogenase in the chromosome. Thus, the microorganism may have the ability to produce 4-hydroxybutyrate (4-HB).

The C4 compound may be an organic acid and may be succinate, fumaric acid, malic acid, or a C4 compound derived therefrom. Also, a material derived from succinate may be 4-hydroxybutyrate (4-HB), 1,4-butanediol (1,4-BDO), γ-butyrolactone (GBL), or a C4 compound derived therefrom, but it is not limited thereto.

The microorganism producing the C4 compound may further include various enzymes. According to an embodiment, the microorganism may further include a polynucleotide coding for a 4-hydroxybutyryl CoA:acetyl-CoA transferase and a polynucleotide coding for an alcohol dehydrogenase. The microorganism including these enzymes may produce 1,4-butanediol.

The 4-hydroxybutyryl CoA:acetyl-CoA transferase may be an enzyme having an activity for catalyzing a reaction that converts 4-hydroxybutyrate into 4-hydroxybutyryl-CoA. The 4-hydroxybutyryl CoA:acetyl-CoA transferase may be an enzyme categorized as EC.2.8.3.a. According to an embodiment, the enzyme may be Cat2. Cat2 may have an amino acid sequence of SEQ ID NO: 14 derived from Porphyromonas gingivalis. The polynucleotide coding for the Cat2 may have a nucleotide sequence of SEQ ID NO: 15.

The alcohol dehydrogenase may be an enzyme having an activity for catalyzing a reaction that converts 4-hydroxybutyryl-CoA into 1,4-butanediol. The alcohol dehydrogenase may be an enzyme categorized as EC.1.1.1.c. According to an embodiment, the enzyme may be AdhE2. The AdhE2 may have an amino acid sequence of SEQ ID NO: 16 derived from Clostridium acetobutylicum. The polynucleotide coding for the AdhE2 may have a nucleotide sequence of SEQ ID NO: 17.

According to another embodiment, provided is a method of producing the C4 compound, the method including: culturing the microorganism to obtain a culture and recovering the C4 compound from the culture.

The microorganism may be a microorganism producing the C4 compound, in which the CoA-dependent succinate semialdehyde dehydrogenase is expressed from a gene in a vector is as described above. Also, the microorganism may additionally include the 4-hydroxybutyrate dehydrogenase in the vector. Also, the microorganism may include a polynucleotide coding for a CoA-dependent succinate semialdehyde dehydrogenase and a polynucleotide coding for a 4-hydroxybutyrate dehydrogenase in a chromosome, apart from the CoA-dependent succinate semialdehyde dehydrogenase and the 4-hydroxybutyrate dehydrogenase expressed in a vector. Also, the microorganism may further include a polynucleotide coding for the 4-hydroxybutyryl CoA:acetyl-CoA transferase and a polynucleotide coding for the alcohol dehydrogenase.

The culturing may be performed in a suitable culture medium and culturing conditions known in the art. One of ordinary skill in the art may easily adjust the culture medium and the culturing conditions according to the selected microorganism. A culturing method may include a batch mode, a continuous mode, a fed-batch mode, or a combination mode thereof.

The culture medium may include various carbon sources, nitrogen sources, and trace element components.

The carbon source may include a carbohydrate such as glucose, sucrose, lactose, fructose, maltose, starch, and cellulose; a lipid such as soybean oil, sunflower oil, castor oil, and coconut oil; a fatty acid such as palmitic acid, stearic acid, and linoleic acid; an alcohol such as glycerol and ethanol; an organic acid such as acetic acid; or a combination thereof. The culturing may be performed by using glucose as a carbon source. The nitrogen source may include an organic nitrogen source such as peptone, yeast extract, meat extract, malt extract, corn steep liquid, and soybean; an inorganic nitrogen source such as urea, ammonium sulfate, ammonium chloride, ammonium phosphate, ammonium carbonate, and ammonium nitrate; or a combination thereof.

The culture medium may include as a phosphorous source, for example, potassium dihydrogen phosphate, dipotassium phosphate, a sodium-containing salt corresponding to potassium dihydrogen phosphate, and dipotassium phosphate, and a metal salt such as magnesium sulfate and iron sulfate. The culture medium or an individual component may be added to the culture in a batch mode or a continuous mode.

In addition, the pH of the culture may be adjusted during the culturing by adding a compound such as ammonium hydroxide, potassium hydroxide, ammonia, phosphoric acid, or sulfuric acid to the culture medium in an appropriate mode. In addition, bubble formation may be repressed by using an anti-foaming agent such as fatty acid polyglycol ester.

The microorganism may be cultured under anaerobic or microaerobic conditions. The wording “microaerobic condition” refers to a state in which the oxygen concentration in the culture medium is lower than that obtained when the culture medium is contacted with the normal atmospheric air. For example, a low level of oxygen can be less than about 10%, 5%, 1%, 0.1%, or 0.01%, about 0.01% to about 10%, about 0.01% to about 5%, about 0.01% to about 1%, about 0.01% to about 0.1%, about 0.1% to about 10%, about 1% to about 9%, about 2% to about 8%, about 3% or 7%, or about 4% to about 6% of the oxygen level in the culture medium obtained when the culture medium allowed to be contacted with the normal atmospheric air.

The microaerobic condition may be formed, for example, by supplying carbon dioxide or nitrogen at a flow rate range from about 0.1 vvm (aeration volume/medium volume/minute) to about 0.4 vvm, from about 0.2 vvm to about 0.3 vvm, or at a flow rate of about 0.25 vvm. Also, the anaerobic or micraerobic condition may have an aeration rate of about 0 vvm (i.e., anaerobic) to about 0.4 vvm, about 0.1 vvm to about 0.3 vvm, and about 0.15 vvm to about 0.25 vvm.

The method includes recovering the produced C4-compound from the culture. The produced C4 compound may be succinate, fumaric acid, malic acid, or a C4 compound derived therefrom. According to an embodiment of the present invention, the microorganism has an enhanced ability to produce the C4 compound of TCA cycle and materials derived therefrom. Examples of succinate derived material may include 4-HB, 1,4-BDO, GBL, or a C4 compound derived therefrom.

According to an embodiment, recovering the 4-hydroxybutyrate may be performed by a well-known separation and purification method. The recovering may be performed by centrifugation, ion exchange chromatography, filtration, precipitation, or a combination thereof.

The recovered produced C4 compound may be converted into another organic compound to further include a process of obtaining various C4 compounds. The 4-hydroxybutyrate produced according to the method described above may be chemically converted to synthesize a substrate that is structurally related thereto. According to an embodiment, 4-hydroxybutyrate may be incubated in the presence of a strong acid at a temperature of about 100° C. to about 200° C. and then distilled to obtain GBL. The obtained GBL may be converted into N-methylpyrrolidone (NMP) through an amination reaction by using an amination agent, for example, methylamine. Also, the obtained GBL may be selectively converted into tetrahydrofuran (THF), 1,4-butanediol, or butanol through hydrogenation by using a metal-containing catalyst, for example, ruthenium (Ru) or palladium (Pd).

Also, the produced 4-hydroxybutyrate may be biologically converted to obtain poly-4-hydroxybutyrate. The biological conversion may be caused by a polyhydroxyalkanoate synthase, 4HB-CoA:CoA transferase, or a combination thereof.

According to an embodiment, provided is a microorganism in which a gene coding for CoA independent SSADH is inactivated or attenuated. The gene coding for CoA independent SSADH may be located in the chromosome.

The microorganism may be any one selected from the group consisting of Lumen bacteria, Corynebacterium genus, and Brevibacterium genus. The Corynebacterium genus microorganism may be a microorganism selected from the group consisting of Corynebacterium glutamicum, Corynebacterium thermoaminogenes, Brevibacterium flavum, and Brevibacterium lactofermentum.

The CoA independent SSADH may have a catalytic activity that converts succinic semialdehde into succinate. The SSADH may be an enzyme categorized as EC.1.2.1.16 or EC.1.2.1.39. The SSADH may be NAD+ dependent or NADP+ dependent. The SSADH may be a polypeptide having an amino acid sequence of SEQ ID NO: 4, SEQ ID NO: 5, or SEQ ID NO: 6. A gene coding for the SSADH may have a nucleotide sequence of SEQ ID NO: 7, SEQ ID NO: 8, or SEQ ID NO: 9.

The microorganism may have at least one inactivated or attenuated polynucleotide selected from a polynucleotide having a nucleotide sequence of SEQ ID NO: 7, a polynucleotide having a nucleotide sequence of SEQ ID NO: 8, and a polynucleotide having a nucleotide sequence of SEQ ID NO: 9. At least one selected from a polynucleotide having a nucleotide sequence of SEQ ID NO: 7 and a polynucleotide having a nucleotide sequence of SEQ ID NO: 8 may be inactivated or attenuated.

The microorganism may be devoid of CoA-independent SSADH activity or may have reduced CoA-independent SSADH activity due to inactivation or attenuation of the gene. The expression “decreased” refers to the activity of a manipulated microorganism in comparison with the activity of an unmanipulated organism, i.e., parent organism.

The microorganism may have an inactivated or attenuated pathway for synthesizing lactate from pyruvate. The microorganism may be devoid of or have decreased activity of L-lactate dehydrogenase (LDH). The LDH may have a catalytic activity for converting pyruvate into lactate. The LDH may be an enzyme categorized as EC.1.1.1.27. For example, the LDH ma have an amino acid sequence of SEQ ID NO: 3. A gene coding for the LDH in the microorganism may be inactivated or may have attenuated activity.

The microorganism may include at least one selected from a polynucleotide coding for succinyl-CoA:coenzyme A transferase (Cat1), a polynucleotide coding for acetyl-CoA hydrolase (ActA and ActB), and a polynucleotide coding for CoA-dependent succinate semialdehyde dehydrogenase (SucD).

The Cat1, ActA, or ActB may have a catalytic activity for converting succinate into succinyl-CoA. The Cat1 may be an enzyme classified as EC.2.8.3.a. For example, the Cat1 may have an amino acid sequence of SEQ ID NO: 12. The polynucleotide coding for Cat1 may have a nucleotide sequence of SEQ ID NO: 13. The ActA may be an enzyme classified as EC.2.8.3.-. For example, the ActA may have an amino acid sequence of SEQ ID NO: 82. The polynucleotide coding for ActA may have a nucleotide sequence of SEQ ID NO: 83. The ActB may be an enzyme classified as EC.2.8.3.8. For example, the ActB may have an amino acid sequence of SEQ ID NO: 84. The polynucleotide coding for ActB may have a nucleotide sequence of SEQ ID NO: 85.

The SucD may have a catalytic activity for converting succinyl-CoA into succinic semialdehyde. The SucD may be an enzyme classified as EC.1.2.1.b. For example, the SucD may have an amino acid sequence of SEQ ID NO: 1. The polynucleotide coding for SucD may have a nucleotide sequence of SEQ ID NO: 2.

The microorganism may further include a polynucleotide coding for 4-hydroxybutyrate dehydrogenase (4hbD). The microorganism may have 4HB productivity.

The 4hbD may have a catalytic activity for converting succinic semialdehyde into 4HB. The 4hbD may be an enzyme classified as EC.1.1.1.a. For example, the 4hbD may have an amino acid sequence of SEQ ID NO: 10. The polynucleotide coding for 4hbD may have a nucleotide sequence of SEQ ID NO: 11.

The microorganism for producing 4HB may be used for producing a convertible material from 4HB. The convertible material may be gamma-butyrolactone (GBL), 1,4-butanediol, poly-4-hydroxybutyrate, tetrahydrofuran (THF), or N-methylpyrrolidone (NMP).

The microorganism may further include a polynucleotide coding for 4-hydroxybutyrate dehydrogenase, a polynucleotide coding for 4-hydroxybutyryl CoA:acetyl-CoA transferase (Cat2), and a polynucleotide coding for alcohol dehydrogenase (AdhE2). The microorganism may capable of producing 1,4-butanediol.

The Cat2 may have a catalytic activity for converting 4HB into 4-hydroxybutyryl-CoA. The Cat2 may be an enzyme classified as EC.2.8.3.a. For example, the Cat2 may have an amino acid sequence of SEQ ID NO: 14. The polynucleotide coding for Cat2 may have a nucleotide sequence of SEQ ID NO: 15 of nucleotide sequence.

The AdhE2 may have a catalytic activity for converting 4-hydroxybutyryl-CoA into 1,4-butanediol. The AdhE2 may be an enzyme classified as EC.1.1.1.c. For example, the AdhE2 may have an amino acid sequence of SEQ ID NO: 16. The polynucleotide coding for AdhE2 may have a nucleotide sequence of SEQ ID NO: 17.

According to another embodiment, provided is a method of producing a C4 compound using the microorganism in which a gene coding for a CoA-independent succinic semialdehyde dehydrogenase is inactivated or attenuated. The microorganism in which a gene coding for a CoA-independent succinic semialdehyde dehydrogenase is inactivated or attenuated is as described above. C4 compound is as described above.

According to another embodiment, provided is a method of producing 4HB, the method including: culturing a microorganism producing 4HB to obtain a culture; and recovering 4HB from the culture.

The 4HB producing microorganism is the same as described above.

The culturing may be performed as described above.

The culturing may occur in anaerobic or a microaerobic condition as described above. The recovering of 4HB may be performed by a separation and purification method known in the art. The recovering may be performed by centrifugation, ion-exchange chromatography, filtration, immersion, or a combination thereof.

According to another embodiment, provided is a method of producing 1,4-butanediol, the method comprising: culturing a microorganism 1,4-butanediol to obtain cultured products; and recovering 1,4-butanediol from the culture.

The microorganism producing 1,4-butanediol is the same as described above.

The culturing is the same as described above. The culturing may occur in anaerobic or a microaerobic condition. The microaerobic condition is the same as described above.

The recovering of 1,4-butanediol may be performed by a separation and purification method known in the art. The recovering may be performed by centrifugation, ion-exchange chromatography, filtration, immersion, or a combination thereof.

Hereinafter, the present invention will be described in further detail with reference to examples. It will be obvious to a person having ordinary skill in the art that these examples are illustrative purposes only and are not to be construed to limit the scope of the present invention.

### Example 1

### Preparation of a Strain with an Inactivated L-Lactate Dehydrogenase Gene

1.1. Preparation of a Replacement Vector

An L-lactate dehydrogenase gene (ldh) of Corynebacterium glutamicum (C. glutamicum, CGL) (ATCC 13032, hereinafter the same) was inactivated through a homologous recombination by using a pK19 mobsacB (ATCC 87098) vector. Two homologous regions for removing the Idh gene were obtained by PCR amplification using a genomic DNA of CGL. The homologous regions were upstream and downstream regions of the Idh gene, each of which was obtained by PCR amplification using a primer set of IdhA_{—}5′_HindIII (SEQ ID NO: 18) and IdhA_up_{—}3′_XhoI (SEQ ID NO: 19) and a primer set of IdhA_dn_{—}5′_XhoI (SEQ ID NO: 20) and IdhA_{—}31 EcoRI (SEQ ID NO: 21). The PCR amplification was performed by repeating 30 cycles each including denaturation at a temperature of 95° C. for 30 seconds, annealing at 55° C. for 30 seconds, and extension at 72° C. for 30 seconds. All the PCR amplifications hereinafter were performed under the same conditions. The amplification products were cloned in the HindIII and EcoRI restriction sites of a pK19 mobsacB vector to prepare a pK19_ΔIdh vector.

1.2. Preparation of a CGL (ΔIdh) Strain

The pK19_ΔIdh vector was introduced to CGL through electroporation. The introduced strain was streaked on a Luria Brain Heart Infusion Supplemented (LBHIS) culture medium containing 25 μg/ml of kanamycin and then cultured at a temperature of 30° C. The LBHIS culture medium included 18.5 g/L of a brain-heart infusion broth, 0.5 M of sorbitol, 5 g/L of bacto-tryptone, 2.5 g/L of bacto-yeast extract, 5 g/L of NaCl, and 18 g/L of bacto agar. Hereinafter, the composition of the LBHIS agar plate was the same. Colonies formed were streaked on an LB-sucrose culture medium, cultured at a temperature of 30° C., and only the colonies in which double crossing-over occurred were selected. Genomic DNA was separated from the selected colonies, a primer set of IdhA up (SEQ ID NO: 22) and IdhA down (SEQ ID NO: 23) was used to confirm the deletion of the Idh gene through PCR. As a result, a CGL (ΔIdh) strain was obtained.

### Example 2

### Preparation of a Strain for Removing a Succinic Semialdehyde Dehydrogenase (ssadh) Gene

2.1. Preparation of a Substitution Vector

A succinic semialdehyde dehydrogenase gene (ssadh) of Corynebacterium glutamicum was inactivated by homologous recombination using a pK19 mobsacB vector. Homologous regions for removing the ssadh gene, NCgl0049 (SEQ ID NO: 27), NCgl0463 (SEQ ID NO: 28), or NCgl2619 (SEQ ID NO: 29) were obtained by PCR amplification using genomic DNA of Corynebacterium glutamicum as a template. Two homologous regions for removing the NCgl0049 gene were upstream and downstream regions of the NCgl0049 gene, which were obtained by PCR amplification using a primer set of 0049-1 for(SEQ ID NO: 24) and 0049-1 rev(SEQ ID NO: 25) and a primer set of 0049-2 for(SEQ ID NO: 26) and 0049-2 rev(SEQ ID NO: 27). The amplification products were cloned in HindIII and PstI restriction sites of the pK19 mobsacB vector to prepare a pK19_Δn0049 vector. Two homologous regions for removing the NCgl0463 gene were upstream and downstream regions of the NCgl0463 gene, which were obtained by PCR amplification using a primer set of 0463-1 for(SEQ ID NO: 28) and 0463-1 rev(SEQ ID NO: 29) and a primer set of 0463-2 for(SEQ ID NO: 30) and 0463-2 rev(SEQ ID NO: 31). The amplification products were cloned in HindIII and PstI restriction sites of the pK19 mobsacB vector to prepare a pK19_Δn0463 vector. Two homologous regions for removing the NCgl2619 gene were upstream and downstream regions of the NCgl2619 gene, which were obtained by PCR amplification using a primer set of 2619-1 for(SEQ ID NO: 32) and 2619-1 rev(SEQ ID NO: 33) and a primer set of 2619-2 for(SEQ ID NO: 34) and 2619-2 rev(SEQ ID NO: 35). The amplification products were cloned in HindIII and PstI restriction sites of the pK19 mobsacB vector to prepare a pK19_Δn2619 vector.

2.2. Preparation of a CGL (ΔIdh Δn0049) Strain, a CGL (ΔIdh Δn0463) Strain, a CGL (ΔIdh Δn2619) Strain, and a B039 Strain

The pK19_Δn0049, pK19_Δn0463, and pK19_Δn2619 vectors were introduced separately or altogether to the CGL (ΔIdh) strain prepared in Example 1 through electroporation. The CGL (ΔIdh) strain to which the pK19_Δn0049, pK19_Δn0463, and pK19_Δn2619 vectors were introduced was streaked in an LBHIS medium containing 25 μg/ml of kanamycin to culture the same at a temperature of 30° C. The colony formed was streaked in an LB-sucrose medium to culture the same at a temperature of 30° C. and only the colonies in which double crossing-over occurred were selected. Genomic DNA was separated from the selected colonies and PCR was performed by using a primer set of 0049for(SEQ ID NO: 36) and 0049rev(SEQ ID NO: 37), a primer set of 0463for(SEQ ID NO: 38) and 0463rev(SEQ ID NO: 39), or a primer set of 2619for(SEQ ID NO: 40) and 2619rev(SEQ ID NO: 41) to confirm the deletion of NCgl0049, NCgl0463, and NCgl2619. Table 1 below shows names and genotypes of the prepared strains.

TABLE 1

| Strain name | Genotype |
|---|---|
| CGL (Δldh Δn0049) | ATCC 13032 Δldh ΔNCgl0049 |
| CGL (Δldh Δn0463) | ATCC 13032 Δldh ΔNCgl0463 |
| CGL (Δldh Δn2619) | ATCC 13032 Δldh ΔNCgl2619 |
| B039 | ATCC 13032 Δldh ΔNCgl0049 ΔNCgl0463 |
| | ΔNCgl2619 |

### Example 3

### Changes in Production of 4HB Depending on the Deletion of a Succinic Semialdehyde Dehydrogenase Gene

3.1. Preparation of a B039 pG3G Strain and Evaluation of 4HB Productivity

3.1.1. Preparation of a pG3G Vector

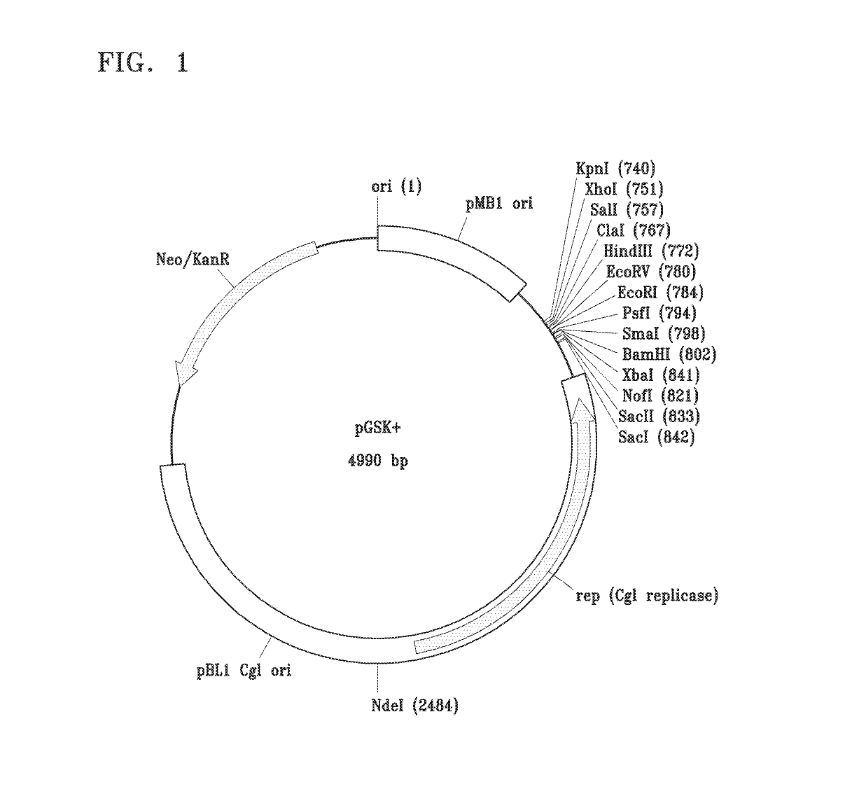

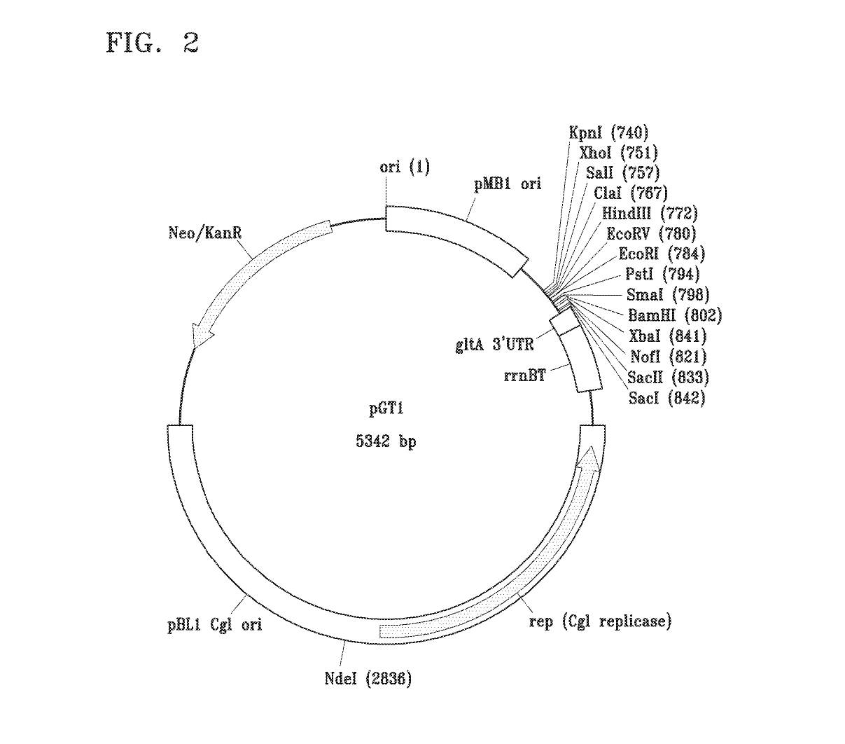

From a pET2 vector (GenBank accession number: AJ885178.1), which is a E. coli-Corynebacterium glutamicum shuttle vector, a region disposed between a replication origin of E. coli and a replication origin of Corynebacterium glutamicum, which is a region including a chloramphenicol acetyltransferase gene and an rrnB transcription terminator, was removed. Also, an aph gene, which is a kanamycin resistance gene, was substituted with a neo gene (nptII), which is a Tn5-derived kanamycin resistance gene, to remove a restriction site and remove an XbaI restriction site in a replicase (rep) gene located in the replication origin of Corynebacterium glutamicum. Thereafter, a multi-cloning site (MCS) derived from a pBlueScript II SK(+) phagemid vector including 14 restriction sites was inserted into the region to prepare a pGSK+ vector (SEQ ID NO: 42). FIG. 1 is a cleavage map of the pGSK+ vector. Based on the pGSK+ vector, 3′UTR and an rrnB transcription terminator of a gltA gene were inserted into a SalI restriction site of pGSK+ to prepare a pGT1 vector (SEQ ID NO: 43). FIG. 2 is a cleavage map of the pGT1 vector.

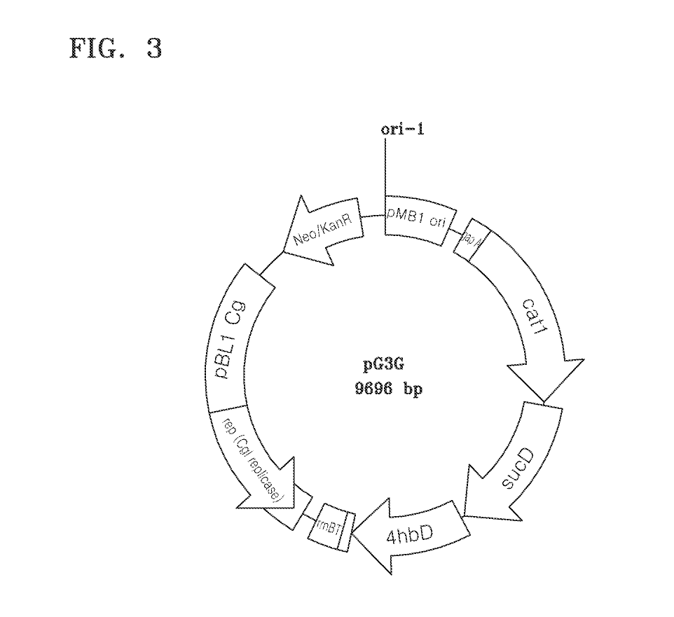

Based on the pGT1 vector, a pG3G, which is an expression vector for cat1, sucD, and 4hbD genes, was prepared. FIG. 3 is a cleavage map of the pG3G vector. A gene cassette (SEQ ID NO: 44), which sequentially includes a gapA promoter, and cat1, sucD, 4hbD, and cat2 genes were prepared. By using the cassette as a template, a sequence between a gapA promoter, which is a promoter for a gapA gene (NCgl1526) derived from Corynebacterium glutamicum, and a 4hbD gene was obtained by PCR amplification using a primer set of 3G up(SEQ ID NO: 45) and 3G down(SEQ ID NO: 46). The amplification product was cloned in PstI and XbaI restriction sites of the pGT1 vector by using an In-fusion HD cloning kit (#PT5162-1, Clontech).

3.1.2. Preparation of a pG2G Vector

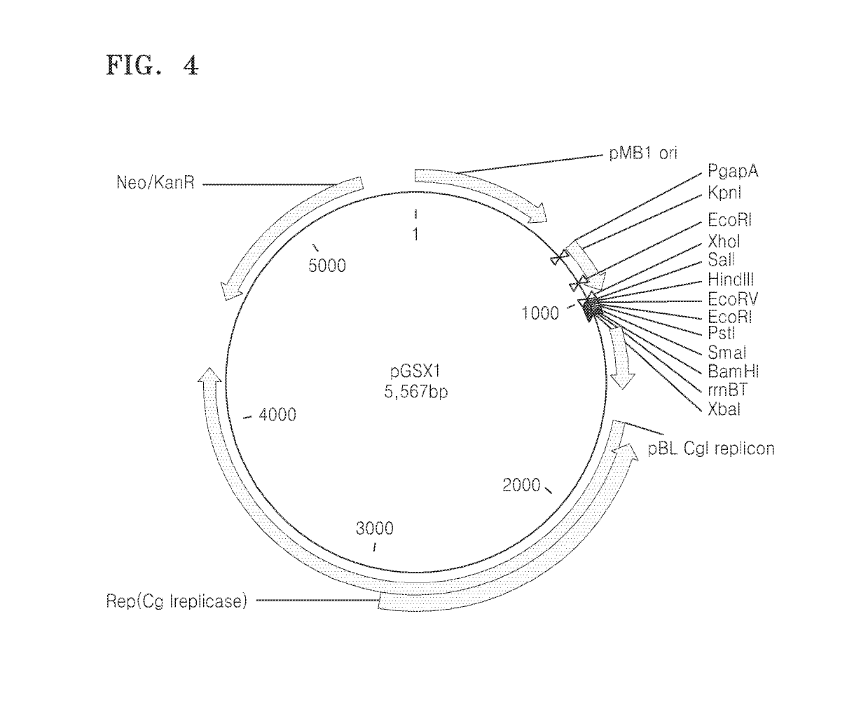

A pGSX1(SEQ ID NO: 47), which is a plasmid vector for expressing cat1, sucD, and 4hbD genes, including a gapA promoter was prepared in the following manner. FIG. 4 is a cleavage map of the pGSX1 vector. The gapA promoter was obtained by PCR amplification using a genomic DNA of Corynebacterium glutamicum ATCC 13032 and a primer set of MD-625 (SEQ ID NO: 48) and MD-626 (SEQ ID NO: 49). The amplification product was cloned by using the In-fusion HD cloning kit, such that a 270 bp DNA fragment was disposed in KpnI and XhoI restriction sites of the pGT1 vector.

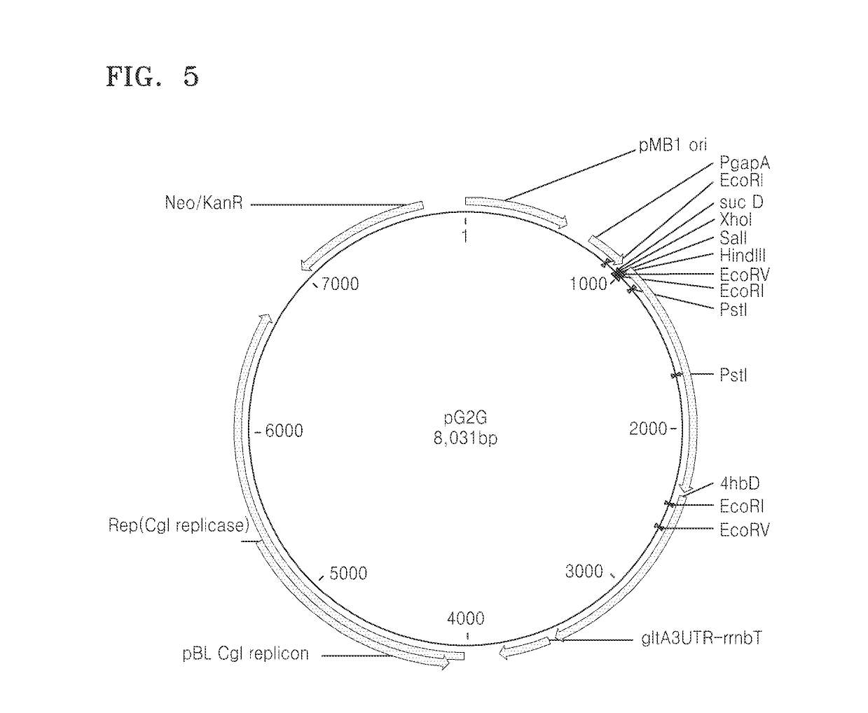

Based on the pGSX1 vector, a pG2G, which is an expression vector for sucD and 4hbD genes, was prepared. FIG. 5 is a cleavage map of the pG2G vector. In the gene cassette, a sucD-4hbD sequence was obtained by PCR amplification using a primer set of sucD-up (SEQ ID NO: 64) and 4hbD-down (SEQ ID NO: 67). The amplification product obtained therefrom was cloned in PstI and XbaI restriction sites of the pGSX1 vector by using the In-fusion HD cloning kit.

3.1.3. Preparation of a Strain

The pG2G and pG3G vectors were introduced into the CGL (ΔIdh) strain or the strain disclosed in Table 1 through electroporation. The introduced strain was streaked in an LBHIS medium containing 25 μg/ml of kanamycin to culture the same at a temperature of 30° C., and strains having kanamycin resistance were selected. Table 2 below shows names and genotypes of the prepared strains.

TABLE 2

| Strain name | Genotype |
|---|---|
| CGL (Δldh pG3G) | ATCC 13032 Δldh pGT1_cat1, sucD, 4hbD |
| CGL (Δldh Δn0049 | ATCC 13032 Δldh ΔNCgl0049 pGT1_cat1, |
| pG3G) | sucD, 4hbD |
| CGL (Δldh Δn0463 | ATCC 13032 Δldh ΔNCgl0463 pGT1_cat1, |
| pG3G) | sucD, 4hbD |
| CGL (Δldh Δn2619 | ATCC 13032 Δldh ΔNCgl2619 pGT1_cat1, |
| pG3G) | sucD, 4hbD |
| B039 pG2G | ATCC 13032 Δldh ΔNCgl0049 ΔNCgl0463 |
| | ΔNCgl2619 pGT1_sucD, 4hbD |
| B039 pG3G | ATCC 13032 Δldh ΔNCgl0049 ΔNCgl0463 |
| | ΔNCgl2619 pGT1_cat1, sucD, 4hbD |

3.1.4. 4HB Production

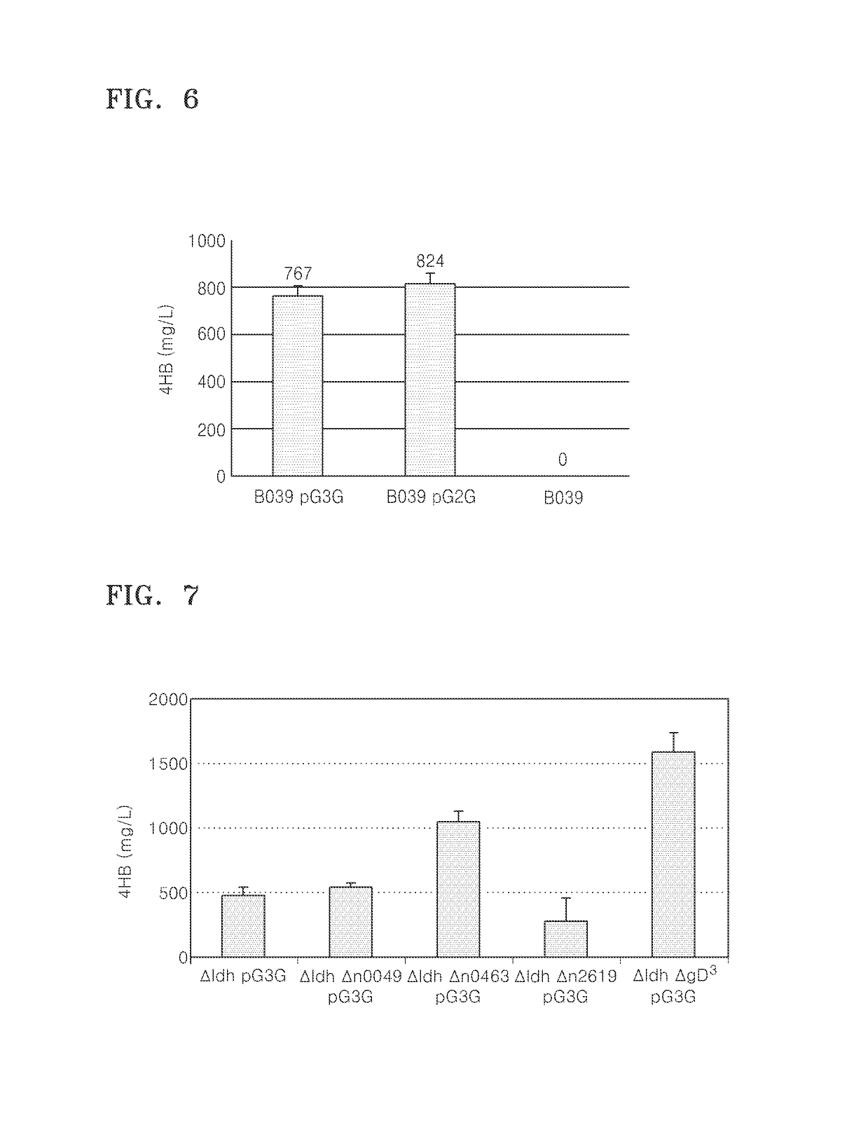

Strains disclosed in Table 2 were cultured in a flask to compare 4HB productivities thereof. The strains were cultured in a brain heart infusion (BHI) medium (Becton, Dickinson and Company) at a temperature of 30° C. and at a rotation speed of 230 rpm for 8 hours. A culture medium solution obtained therefrom was inoculated in a CgXII medium (Keilhauer et al. J. Bacteriol. 1993, 175:5595) at a concentration of 1/50, and then cultured at 30° C. and at 230 rpm for 16 hours. Cap and neck portions of the flask were sealed to create a microaerobic condition and then cultured at 30° C. and at 230 rpm for 8 hours (FIG. 6) or 24 hours (FIG. 7).

FIG. 6 shows 4HB production of B039 pG3G and B039 pG2G. FIG. 7 shows measurement results of 4HB production after introducing a pG3G or a pG2G into 4HB production of the strains described in Table 2. FIG. 6 shows measurement results of 4HB production after introducing the pG3G or the pG2G vector in which all three types of ssadh genes were partially or completely deleted. FIG. 7 shows measurement results of 4HB production after introducing the pG3G vector in which all three types of ssadh genes were partially or completely deleted. The B039 pG3G strain, in which all three types of ssadh genes present in the chromosome were deleted, showed at least a 230% increase in 4HB production, compared to the CGL (ΔIdh pG3G) strain in which all the three types of ssadh genes present in the chromosome were not deleted.

3.2. Preparation of a D003 Strain and Evaluation of 4HB Productivity

3.2.1. Preparation of a pK19 gapA::3G Vector

Based on the pK19 mobsacB, a pK19 gapA::3G vector for inserting cat1, sucD, and 4hbD genes of a Corynebacterium chromosome was prepared. Two homologous regions for homologous recombination were obtained by PCR amplification using a genomic DNA of Corynebacterium glutamicum ATCC 13032 and a primer set of ncgl0049_up F(SEQ ID NO: 50) and ncgl0049_up R(SEQ ID NO: 51) and a primer set of ncgl0049_down F(SEQ ID NO: 52) and ncgl0049_down R(SEQ ID NO: 53), respectively. A DNA fragment, including a gapA promoter and cat1, sucD and 4hbD genes, was obtained by PCR amplification using a pG3G vector as a template and a primer set of pG3G F(SEQ ID NO: 54) and pG3G R(SEQ ID NO: 55). In HindIII and EcoRI restriction sites of the pK19 mobsacB vector, the amplification product was cloned by using the In-fusion HD cloning kit, such that the DNA fragment including the gapA promoter and cat1, sucD and 4hbD genes was disposed between the two homologous regions, to prepare a pK19 gapA::3G vector.

3.2.2. Preparation of a Strain

The pK19 gapA::3G vector was introduced to a CGL (ΔIdh) strain or a CGL (ΔIdh ΔgD3) strain through electroporation. The introduced strain was streaked in an LBHIS medium containing 25 μg/ml of kanamycin to culture the same at a temperature of 30° C. Then, the colonies formed were streaked in an LB-sucrose medium to culture the same at 30° C. and then only the colonies, in which a double crossing-over occurred, were selected. Genomic DNA was separated from the selected colonies and introduction of cat1, sucD, and 4hbD genes were confirmed by using a primer set of cat1-seq1F(SEQ ID NO: 56) and cat1-seq2R(SEQ ID NO: 57), a primer set of sucD-seq1F(SEQ ID NO: 58) and sucD-seq2R(SEQ ID NO: 59), and a primer set of 4hbD-seq1F(SEQ ID NO: 60) and 4hbD-seq2R(SEQ ID NO: 61), respectively. Table 3 below shows strain names and genotypes of the prepared strains.

TABLE 3

| Strain name | Genotype |
|---|---|
| D001 | ATCC 13032 Δldh, gapA::cat1, sucD, 4hbD |
| D003 | ATCC 13032 Δldh ΔNCgl0049 ΔNCgl0463 |
| | ΔNCgl2619, gapA::cat1, sucD, 4hbD |

3.2.3. 4HB Production

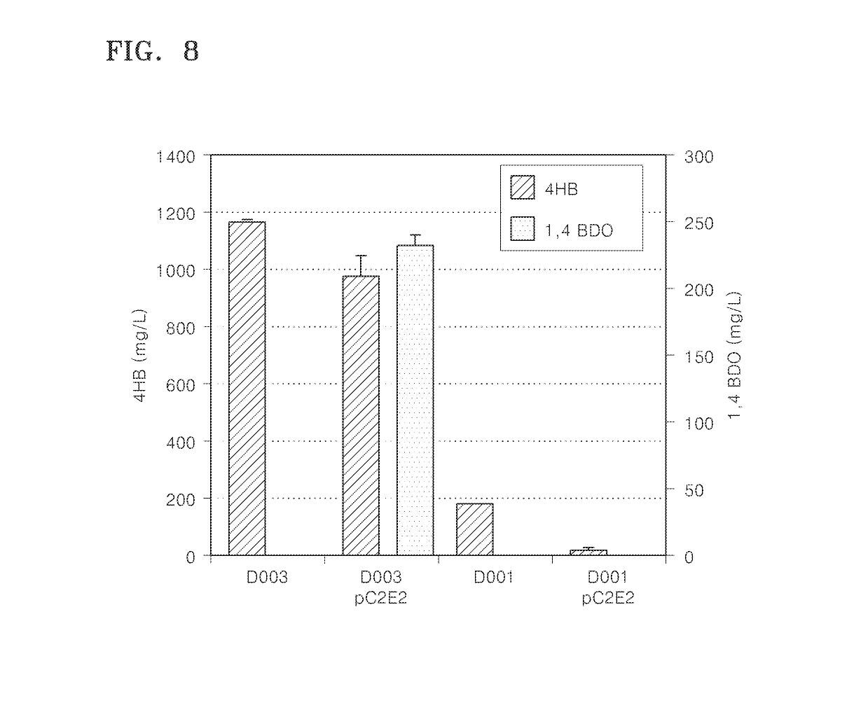

The strains disclosed in Table 3 were cultured in the same manner as in Example 3.1. The final culture medium solution was centrifuged, filtered, and then analyzed for 4HB by using ultra performance liquid chromatography (UPLC). The UPLC analysis was performed with reference to a conventional method (J Chromatogra.B 885-886, 2012, 37-42). An apparatus used for the UPLC analysis was an Acquity UPLC (Waters) loaded with an HSS T3 column (2.1 mm id×100 mm, dp=1.7 μm). As a mobile phase, 0.2% formic acid (A) and methanol (B) were used, the HSS T3 column was maintained at a temperature of 65° C., and chromatography was performed at a flow rate of 0.5 ml/min. As shown in FIG. 8, D003 strain, in which all three types of ssadh genes present in the chromosome were deleted, produced about 1160 mg/L and thus, showed 4HB production that is about 6.4 times as great as D001 strain in which the genes were not deleted.

3.3. Preparation of a D003 pG3G Strain and Evaluation of 4HB Productivity

3.3.1. Preparation of a Vector

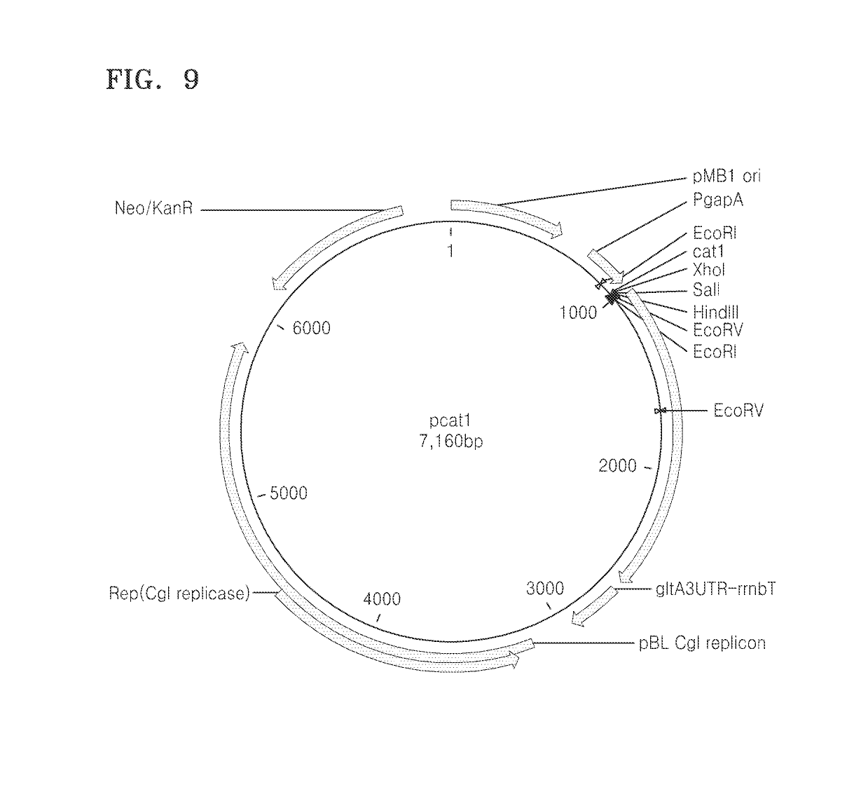

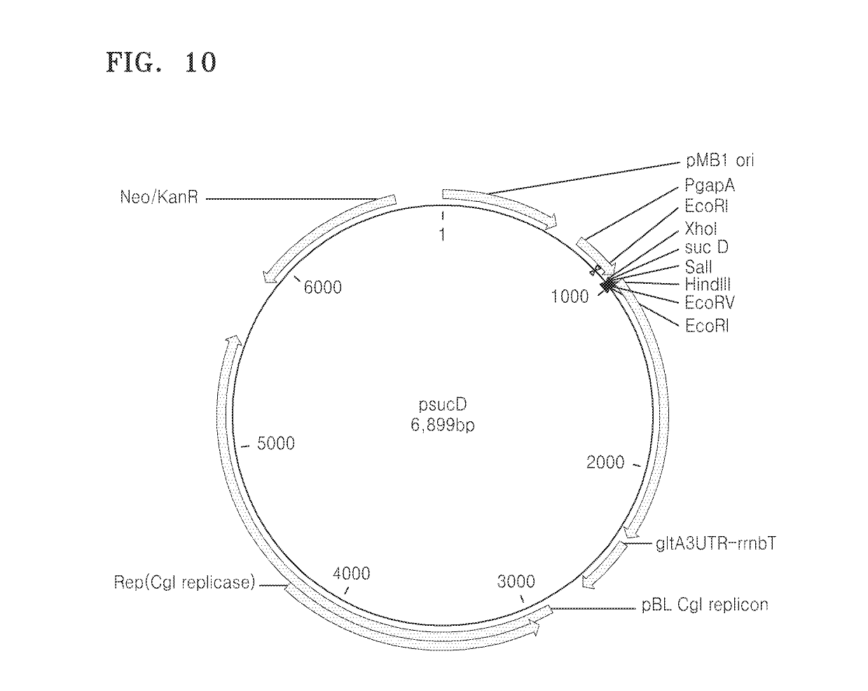

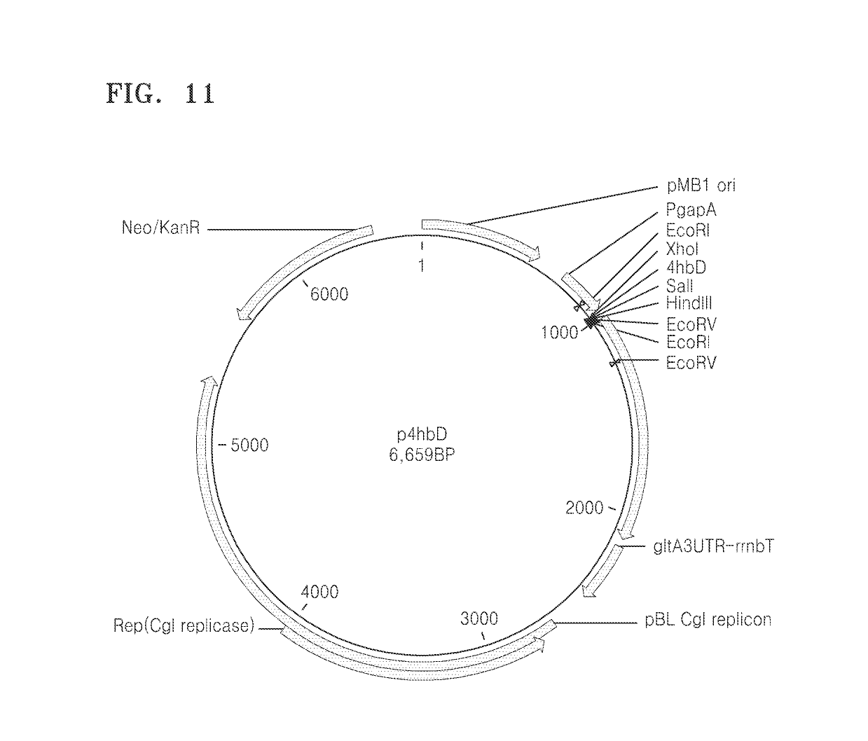

Based on the pGSX1 vector, expression vectors, pcat1, psucD, and p4hbD were prepared for cat1, sucD, and 4hbD genes, respectively. FIGS. 9, 10, and 11 show cleavage maps of pcat1, psucD, and p4hbD vectors. In the gene cassette, the cat1 sequence was obtained by PCR amplification using a primer set of cat1-up (SEQ ID NO: 62) and cat1-down (SEQ ID NO: 63). The sucD sequence was obtained by PCR amplification using a primer set of sucD-up(SEQ ID NO: 64) and sucD-down(SEQ ID NO: 65). The 4hbD sequence was obtained by PCR amplification using a primer set of 4hbD-up (SEQ ID NO: 66) and 4hbD-down (SEQ ID NO: 67). The amplification products obtained therefrom were cloned in PstI and XbaI restriction sites of the pGSX1 vector by using the In-fusion HD cloning kit.

3.3.2. Preparation of a Strain

The pcat1, psucD, p4hbD, pG2G, and pG3G vectors were introduced to the D003 strain through electroporation. Table 4 shows the genotypes of the prepared strains. The introduced strains were cultured in an LB medium and then freeze-stored.

TABLE 4

| Strain names | Genotypes |
|---|---|
| D003 | ATCC13032 Δldh ΔNCgl0049 ΔNCgl0463 ΔNCgl2619, |
| | gapA::cat1:sucD:4hbD |
| D003 pcat1 | ATCC13032 Δldh ΔNCgl0049 ΔNCgl0463 ΔNCgl2619, |
| | gapA::cat1:sucD:4hbD, pcat1 |
| D003 psucD | ATCC13032 Δldh ΔNCgl0049 ΔNCgl0463 ΔNCgl2619, |
| | gapA::cat1:sucD:4hbD, psucD |
| D003 p4hbD | ATCC13032 Δldh ΔNCgl0049 ΔNCgl0463 ΔNCgl2619, |
| | gapA::cat1:sucD:4hbD, p4hbD |
| D003 pG2G | ATCC13032 Δldh ΔNCgl0049 ΔNCgl0463 ΔNCgl2619, |
| | gapA::cat1:sucD:4hbD, pG2G |
| D003 pG3G | ATCC13032 Δldh ΔNCgl0049 ΔNCgl0463 ΔNCgl2619, |
| | gapA::cat1:sucD:4hbD, pG3G |

3.3.3. Production of 4HB

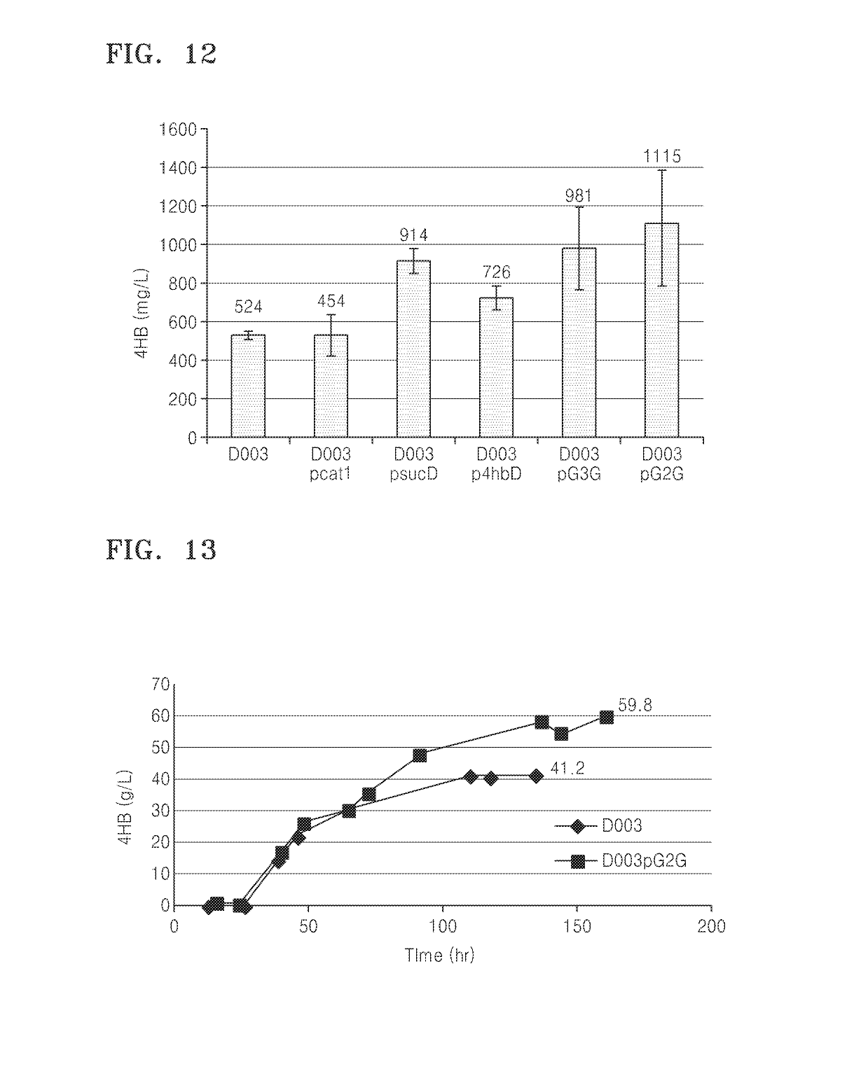

The strains disclosed in Table 4 were cultured for 24 hours in a flask in the same method as in Example 3.1. to compare 4HB productivity. FIG. 12 shows an amount of 4HB produced in a culture using the strains disclosed in Table 4. As shown in FIG. 12, 4HB productivity of the D003 pG2G was the greatest.

With respect to the strains, 4HB productivities thereof were compared through a fermentation experiment. The fermentation strains were cultured in a medium including 4% of glucose, 1% of polypeptone, 5 g/L of yeast extract, 2 g/L of (NH₄)₂SO₄, 4 g/L of potassium phosphate monobasic, 8 g/L of potassium phosphate dibasic, 0.5 g/L of MgSO4.7H₂O, 1 mg/L of thiamine-HCl, 0.1 mg/L of D-biotin, and trace elements (0.1-10 ppm of iron, manganese, zinc, copper) at a temperature of 30° C. and at a speed of 230 rpm for about 8 hours to about 12 hours. The fermentation medium included 1% of CSL, 2 g/L of (NH₄)₂SO₄, 1 g/L of potassium phosphate monobasic, 0.5 g/L of MgSO4.7H2O, 3 mg/L of thiamine-HCl, 0.3 mg/L of D-biotin, and trace elements (0.1˜10 ppm of iron, manganese, zinc, and copper), and glucose was added according to an amount of glucose consumption during the fermentation. The strains were cultured under conditions of culturing temperature at about 30° C. to about 35° C., stirring speed of about 500 rpm to about 800 rpm, amount of air current of about 0.5 vvm to about 2 vvm, pH during culturing of about 6.5 to about 8.0 in an aerobic culture for about 17 hours to about 35 hours, and then switched to anaerobic or microaerobicconditions under the same culturing temperature and pH during culturing, to culture the strains at a stirring speed of about 100 rpm to about 500 rpm and amount of air current of about 0 vvm to about 0.4 vvm for about 24 hours to about 150 hours. FIG. 13 shows a production amount of 4HB in D003 and D003 pG2G. As shown in FIG. 13, it was confirmed that D003 pG2G, which increased the expression of sucD and 4hbD, increased 4HB productivity to a level that is about 1.5 times as great as that of D003. For FIG. 13, D003 and D003 pG2G were cultured at 30° C. for 24 hours in aerobic conditions and then cultured in a microaerobic conditions, i.e., with an aeration rate of 0.1 vvm.

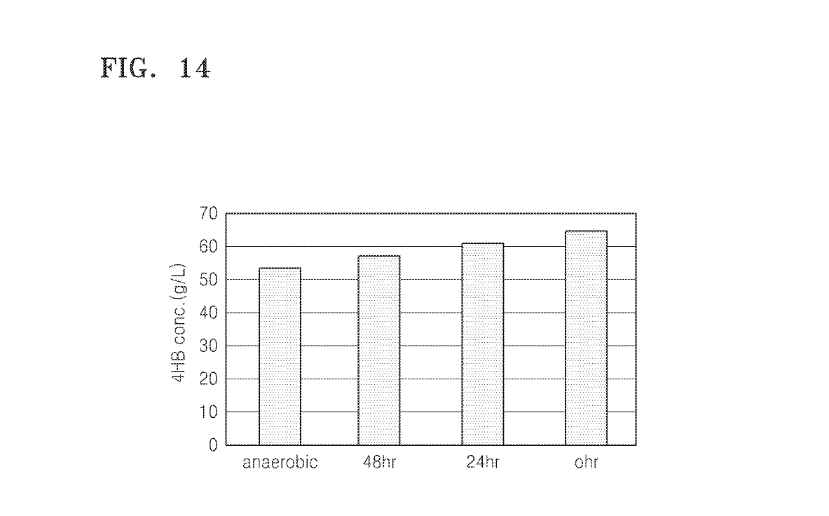

Also, during the fermentation process of D003 pG2G, 4HB productivities according to changes in air flow conditions were compared after performing the aerobic culturing as described above. After the completion of aerobic growth, 4HB productivities were measured for a case in which the strains were maintained in a microaerobic state immediately thereafter at 0.1 vvm and a case in which the strains were maintained in an anaerobic condition without aeration, and a case in which the strains were maintained in anaerobic conditions for 24 hours to 48 hours and then maintained in an microaerobic state at 0.1 vvm. FIG. 14 shows 4HB productivities according to aeration conditions after growth by aerobic culturing. In FIG. 14, ‘0 hr’ represents the case in which the microaerobic state was maintained at 0.1 vvm immediately after the completion of the aerobic growth for 114 hours. The expression, ‘anaerobic’ represents the case in which the strains were maintained without aeration, i.e., the strains were maintained in a closed vessel without aeration, thus the oxygen is only provided from the head space of the vessel and the expressions ‘48 hr’ and ‘24 hr’ represent the case in which the strains were maintained in anaerobic conditions for 24 hours to 48 hours and then maintained in an microaerobic state at 0.1 vvm to the end. As a result, as shown in FIG. 14, ‘0 hr’ showed 4HB productivity that is 1.2 times as great as that of the case in which the strains were maintained in an anaerobic condition.

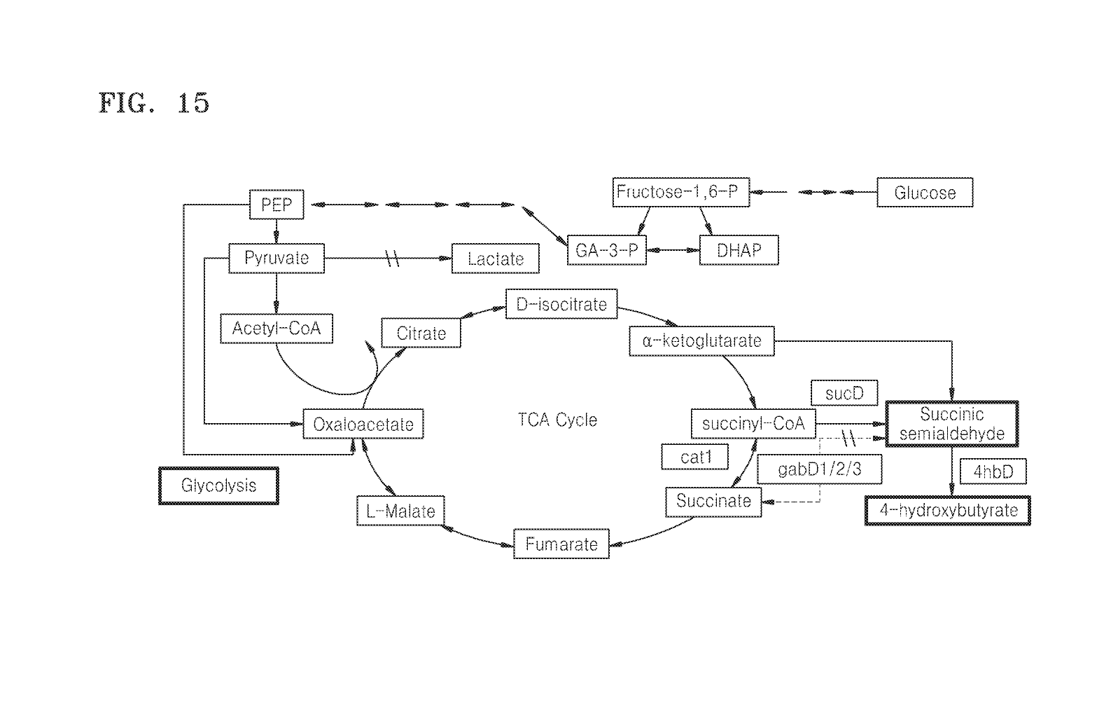

FIG. 15 shows an example of a biosynthetic pathway for producing 4-hydroxybutyrate from glucose

### Example 4

### Changes in 1,4-BDO Production Due to Deletion of a Succinic Semialdehyde Dehydrogenase Gene

4.1. Preparation of a D003 pC2E2 Strain

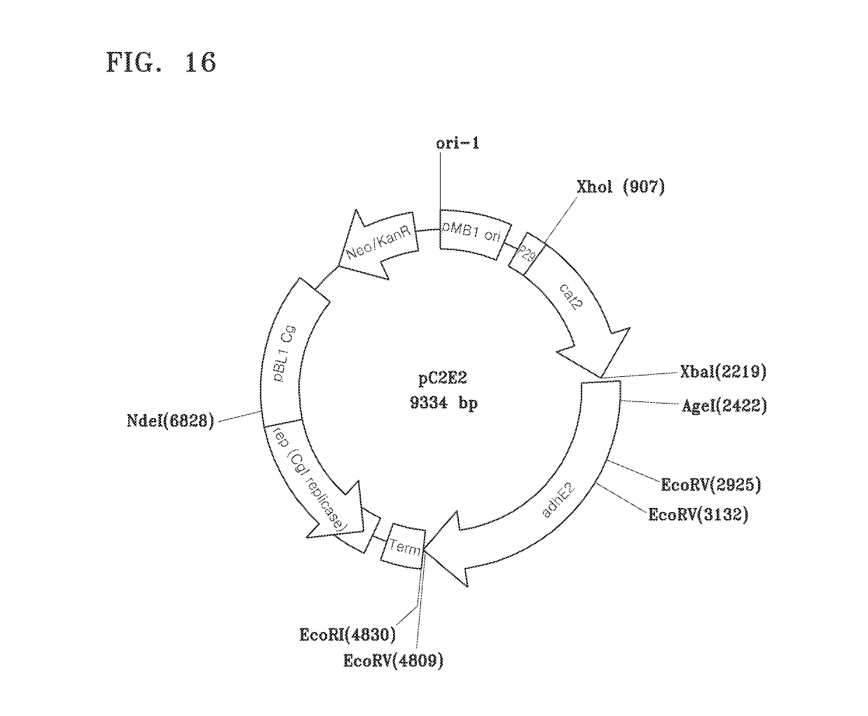

Based on the pGSK+ vector, cat2 and adhE2 gene expression vector pC2E2 (SEQ ID NO: 68) was prepared. FIG. 16 is a cleavage map of a pC2E2 vector. A P29 promoter (P29) was obtained by PCR amplification using a genomic DNA of Corynebacterium glutamicum ATCC 13032 as a template and a primer set of P1(SEQ ID NO: 69) and P2(SEQ ID NO: 70). The cat2 gene was obtained by PCR amplification using a vector MD0294(SEQ ID NO: 71) as a template and a primer set of P3(SEQ ID NO: 72) and P4(SEQ ID NO: 73). The adhE2 gene was obtained by PCR amplification using a vector MD0295(SEQ ID NO: 74) as a template and a primer set of P5(SEQ ID NO: 75) and P6(SEQ ID NO: 76). A transcription terminator region (Term) was obtained by using a vector pGSX1 as a template and a primer set of P7(SEQ ID NO: 77) and P8(SEQ ID NO: 78). The amplification products obtained therefrom were cloned in KpnI and SacI restriction sites of the pGSK+ vector by using the In-fusion PCR cloning kit.

The pC2E2 vector was introduced into the strains D001 and D003 prepared in Example 3.1. by using electroporation. The introduced strains were streaked in an LBHIS medium containing 25 μg/ml of kanamycin to culture the same at a temperature of 30° C., and strains having kanamycin resistance were selected. Table 5 below shows strain names and genotypes of the prepared strains.

TABLE 5

| Strain name | Genotype |
|---|---|
| D001 pC2E2 | ATCC 13032 Δldh, gapA::cat1, sucD, 4hbD, |
| | pGSK+_cat2, adhE2 |
| D003 pC2E2 | ATCC 13032 Δldh ΔNCgl0049 ΔNCgl0463 ΔNCgl2619, |
| | gapA::cat1, sucD, 4hbD, pGSK+_cat2, adhE2 |

4.3. Production of 1,4-BDO

The D001 pC2E2 and D003 pC2E2 strains disclosed in Table 5 were cultured under the same conditions as described in Example 3.1, and then 1,4-BDO was analyzed by using UPLC. As shown in FIG. 8, three types of ssadh genes were deleted and the D003 pC2E2 strain in which cat2 and adhE2 genes were additionally introduced produced about 232.5 mg/L of 1,4-BDO. On the contrary, the D001 pC2E2 strain in which the ssadh gene was not deleted did not show 1,4-BDO production. The D003 pC2E2 strain in which three types of ssadh genes were deleted may be effectively used for 1,4-BDO production.

As described above, according to the one or more of the above embodiments of the present invention, a microorganism producing a C4 compound in which a CoA-dependent succinate semialdehyde dehydrogenase is expressed in a vector may produce the C4 compound more effectively. By using the microorganism, the efficiency of the C4 compound production may increase to produce the C4 compound that may be used for various industrial purposes.

The microorganism in which SSADH gene present in the chromosome is inactivated or attenuated according to an embodiment may be used for the production of useful metabolites.

According to a method of producing 4-hydroxybutyrate according to another embodiment may effectively produce 4-hydroxybutyrate.

According to a method of producing 1,4-butanediol according to another embodiment may effectively produce 1,4-butanediol.

It should be understood that the exemplary embodiments described therein should be considered in a descriptive sense only and not for purposes of limitation. Descriptions of features or aspects within each embodiment should typically be considered as available for other similar features or aspects in other embodiments.

While one or more embodiments of the present invention have been described with reference to the figures, it will be understood by those of ordinary skill in the art that various changes in form and details may be made therein without departing from the spirit and scope of the present invention as defined by the following claims.

What is claimed is:

1. A microorganism comprising a vector encoding a CoA-dependent succinate semialdehyde dehydrogenase, wherein the microorganism expresses the CoA-dependent succinate semialdehyde dehydrogenase from the vector and produces a C4 compound.

2. The microorganism of claim 1, wherein the microorganism belongs to a Corynebacterium genus.

3. The microorganism of claim 1, wherein the microorganism comprises a gene coding for an L-lactate dehydrogenase that is inactivated or attenuated.

4. The microorganism of claim 1, wherein the microorganism comprises a gene coding for a genomic CoA-independent succinic semialdehyde dehydrogenase that is inactivated or attenuated.

5. The microorganism of claim 4, wherein the CoA-independent succinic semialdehyde dehydrogenase has an amino acid sequence of SEQ ID NO: 4, SEQ ID NO: 5, or SEQ ID NO: 6.

6. The microorganism of claim 1, wherein the microorganism further comprises a vector encoding a 4-hydroxybutyrate dehydrogenase, and the microorganism expresses 4-hydroxybutyrate dehydrogenase from the vector.

7. The microorganism of claim 1, wherein the microorganism comprises a polynucleotide coding for a CoA-dependent succinate semialdehyde dehydrogenase and a polynucleotide coding for a 4-hydroxybutyrate dehydrogenase in a chromosome of the microorganism.

8. The microorganism of claim 1, wherein the microorganism further comprises a polynucleotide coding for a succinyl-CoA:CoA transferase.

9. The microorganism of claim 8, wherein the polynucleotide coding for a succinyl-CoA:CoA transferase is in a chromosome of the microorganism.

10. The microorganism of claim 1, wherein the C4 compound is 4-hydroxybutyrate (4-HB).

11. The microorganism of claim 1, wherein the vector is a plasmid.

12. The microorganism of claim 1, wherein the microorganism further comprises a polynucleotide coding for a 4-hydroxybutyrate dehydrogenase, a polynucleotide coding for a 4-hydroxybutyryl CoA:acetyl-CoA transferase, and a polynucleotide coding for an alcohol dehydrogenase.

13. A method of producing a C4 compound, the method comprising:
culturing the microorganism of claim 1 with a carbon source; and
recovering the C4 compound from the culture.

14. The method of claim 13, wherein the C4 compound is succinate, fumaric acid, malic acid, 4-hydroxybutyrate, 1,4-butanediol, γ-butyrolactone, or C4 compounds derived therefrom.

15. The method of claim 13, wherein the microorganism is cultured under aerobic conditions and subsequently cultured under anaerobic conditions.

16. The method of claim 13, wherein the microorganism is cultured under aerobic conditions and subsequently cultured under microaerobic conditions.

17. The method of claim 16, wherein culturing under microaerobic conditions comprises providing carbon dioxide or nitrogen to the culture at a rate of about 0.1 vvm to about 0.4 vvm.

18. The method of claim 15, further comprising culturing the microorganism under microaerobic conditions 0 to 24 hours after culturing the microorganism under anaerobic conditions.

19. A Corynebacterium genus microorganism in which a gene coding for a CoA-independent succinic semialdehyde dehydrogenase is inactivated or attenuated, and L-lactate hydrogenase activity is eliminated or attenuated,
wherein the microorganism comprises at least one selected from a polynucleotide coding for a succinyl CoA transferase, a polynucleotide coding for acetyl-CoA hydrolase, and a polynucleotide coding for a CoA-dependent succinate semialdehyde dehydrogenase, and
wherein the microorganism further comprises a polynucleotide coding for a 4-hydroxybutyrate dehydrogenase (4hbD).

20. A method of producing 4-hydroxybutyrate, the method comprising:
culturing the microorganism of claim 19 with a carbon source; and
recovering 4-hydroxybutyrate from the culture.
